SEQUENCE LISTING (207 sequences)

SEQ ID NO: 1 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, synthetic construct
  REGION 1..141: note = Description of Artificial Sequence: Syntheticpolypeptide
MFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 3 - protein, 15 aa, synthetic construct
  REGION 1..15: note = Description of Artificial Sequence: Syntheticpeptide
MAEGEITTFTALTEK

SEQ ID NO: 4 - protein, 5 aa, synthetic construct
  REGION 1..5: note = Description of Artificial Sequence: Syntheticpeptide
ALTEK

SEQ ID NO: 5 - protein, 4 aa, synthetic construct
  REGION 1..4: note = Description of Artificial Sequence: Syntheticpeptide
LTEK

SEQ ID NO: 6-8 - skipped (3 entries)

SEQ ID NO: 9 - protein, 146 aa, synthetic construct
  REGION 1..146: note = Description of Artificial Sequence: Syntheticpolypeptide
MALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 10 - protein, 145 aa, synthetic construct
  REGION 1..145: note = Description of Artificial Sequence: Syntheticpolypeptide
MLTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - protein, 144 aa, synthetic construct
  REGION 1..144: note = Description of Artificial Sequence: Syntheticpolypeptide
MTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 12 - protein, 143 aa, synthetic construct
  REGION 1..143: note = Description of Artificial Sequence: Syntheticpolypeptide
MEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 13 - protein, 142 aa, synthetic construct
  REGION 1..142: note = Description of Artificial Sequence: Syntheticpolypeptide
MKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 14 - protein, 146 aa, synthetic construct
  REGION 1..146: note = Description of Artificial Sequence: Syntheticpolypeptide
MALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 15 - protein, 145 aa, synthetic construct
  REGION 1..145: note = Description of Artificial Sequence: Syntheticpolypeptide
MLTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 16 - protein, 144 aa, synthetic construct
  REGION 1..144: note = Description of Artificial Sequence: Syntheticpolypeptide
MTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 17 - protein, 143 aa, synthetic construct
  REGION 1..143: note = Description of Artificial Sequence: Syntheticpolypeptide
MEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 18 - protein, 142 aa, synthetic construct
  REGION 1..142: note = Description of Artificial Sequence: Syntheticpolypeptide
MKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 19 - protein, 145 aa, synthetic construct
  REGION 1..145: note = Description of Artificial Sequence: Syntheticpolypeptide
ALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 20 - protein, 144 aa, synthetic construct
  REGION 1..144: note = Description of Artificial Sequence: Syntheticpolypeptide
LTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 21 - protein, 143 aa, synthetic construct
  REGION 1..143: note = Description of Artificial Sequence: Syntheticpolypeptide
TEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 22 - protein, 142 aa, synthetic construct
  REGION 1..142: note = Description of Artificial Sequence: Syntheticpolypeptide
EKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 23 - protein, 141 aa, synthetic construct
  REGION 1..141: note = Description of Artificial Sequence: Syntheticpolypeptide
KFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 24 - protein, 145 aa, synthetic construct
  REGION 1..145: note = Description of Artificial Sequence: Syntheticpolypeptide
ALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 25 - protein, 144 aa, synthetic construct
  REGION 1..144: note = Description of Artificial Sequence: Syntheticpolypeptide
LTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 26 - protein, 143 aa, synthetic construct
  REGION 1..143: note = Description of Artificial Sequence: Syntheticpolypeptide
TEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 27 - protein, 142 aa, synthetic construct
  REGION 1..142: note = Description of Artificial Sequence: Syntheticpolypeptide
EKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 28 - protein, 141 aa, synthetic construct
  REGION 1..141: note = Description of Artificial Sequence: Syntheticpolypeptide
KFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 29 - protein, 139 aa, synthetic construct
  REGION 1..139: note = Description of Artificial Sequence: Syntheticpolypeptide
NLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 30 - protein, 138 aa, synthetic construct
  REGION 1..138: note = Description of Artificial Sequence: Syntheticpolypeptide
LPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 31 - protein, 137 aa, synthetic construct
  REGION 1..137: note = Description of Artificial Sequence: Syntheticpolypeptide
PPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 32 - protein, 136 aa, synthetic construct
  REGION 1..136: note = Description of Artificial Sequence: Syntheticpolypeptide
PGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 33 - protein, 139 aa, synthetic construct
  REGION 1..139: note = Description of Artificial Sequence: Syntheticpolypeptide
NLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 34 - protein, 138 aa, synthetic construct
  REGION 1..138: note = Description of Artificial Sequence: Syntheticpolypeptide
LPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 35 - protein, 137 aa, synthetic construct
  REGION 1..137: note = Description of Artificial Sequence: Syntheticpolypeptide
PPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 36 - protein, 136 aa, synthetic construct
  REGION 1..136: note = Description of Artificial Sequence: Syntheticpolypeptide
PGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 37 - protein, 140 aa, synthetic construct
  REGION 1..140: note = Description of Artificial Sequence: Syntheticpolypeptide
MNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 38 - protein, 139 aa, synthetic construct
  REGION 1..139: note = Description of Artificial Sequence: Syntheticpolypeptide
MLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 39 - protein, 138 aa, synthetic construct
  REGION 1..138: note = Description of Artificial Sequence: Syntheticpolypeptide
MPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 40 - protein, 137 aa, synthetic construct
  REGION 1..137: note = Description of Artificial Sequence: Syntheticpolypeptide
MPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 41 - protein, 140 aa, synthetic construct
  REGION 1..140: note = Description of Artificial Sequence: Syntheticpolypeptide
MNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 42 - protein, 139 aa, synthetic construct
  REGION 1..139: note = Description of Artificial Sequence: Syntheticpolypeptide
MLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 43 - protein, 138 aa, synthetic construct
  REGION 1..138: note = Description of Artificial Sequence: Syntheticpolypeptide
MPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 44 - protein, 137 aa, synthetic construct
  REGION 1..137: note = Description of Artificial Sequence: Syntheticpolypeptide
MPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 45 - protein, 145 aa, synthetic construct
  REGION 1..145: note = Description of Artificial Sequence: Syntheticpolypeptide
MALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 46 - protein, 144 aa, synthetic construct
  REGION 1..144: note = Description of Artificial Sequence: Syntheticpolypeptide
MALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 47 - protein, 144 aa, synthetic construct
  REGION 1..144: note = Description of Artificial Sequence: Syntheticpolypeptide
MALTEKPPGGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 48 - protein, 142 aa, synthetic construct
  REGION 1..142: note = Description of Artificial Sequence: Syntheticpolypeptide
MALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 49 - protein, 145 aa, synthetic construct; identical to SEQ ID NO: 10
  REGION 1..145: note = Description of Artificial Sequence: Syntheticpolypeptide

SEQ ID NO: 50 - protein, 144 aa, synthetic construct
  REGION 1..144: note = Description of Artificial Sequence: Syntheticpolypeptide
MLTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 51 - protein, 143 aa, synthetic construct
  REGION 1..143: note = Description of Artificial Sequence: Syntheticpolypeptide
MLTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 52 - protein, 142 aa, synthetic construct
  REGION 1..142: note = Description of Artificial Sequence: Syntheticpolypeptide
MLTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 53 - protein, 141 aa, synthetic construct
  REGION 1..141: note = Description of Artificial Sequence: Syntheticpolypeptide
MLTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 54 - protein, 144 aa, synthetic construct; identical to SEQ ID NO: 11
  REGION 1..144: note = Description of Artificial Sequence: Syntheticpolypeptide

SEQ ID NO: 55 - protein, 143 aa, synthetic construct
  REGION 1..143: note = Description of Artificial Sequence: Syntheticpolypeptide
MTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 56 - protein, 142 aa, synthetic construct
  REGION 1..142: note = Description of Artificial Sequence: Syntheticpolypeptide
MTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 57 - protein, 141 aa, synthetic construct
  REGION 1..141: note = Description of Artificial Sequence: Syntheticpolypeptide
MTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 58 - protein, 140 aa, synthetic construct
  REGION 1..140: note = Description of Artificial Sequence: Syntheticpolypeptide
MTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 59 - protein, 143 aa, synthetic construct; identical to SEQ ID NO: 12
  REGION 1..143: note = Description of Artificial Sequence: Syntheticpolypeptide

SEQ ID NO: 60 - protein, 142 aa, synthetic construct
  REGION 1..142: note = Description of Artificial Sequence: Syntheticpolypeptide
MEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 61 - protein, 141 aa, synthetic construct
  REGION 1..141: note = Description of Artificial Sequence: Syntheticpolypeptide
MEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 62 - protein, 140 aa, synthetic construct
  REGION 1..140: note = Description of Artificial Sequence: Syntheticpolypeptide
MEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 63 - protein, 139 aa, synthetic construct
  REGION 1..139: note = Description of Artificial Sequence: Syntheticpolypeptide
MEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 64 - protein, 142 aa, synthetic construct; identical to SEQ ID NO: 13
  REGION 1..142: note = Description of Artificial Sequence: Syntheticpolypeptide

SEQ ID NO: 65 - protein, 141 aa, synthetic construct
  REGION 1..141: note = Description of Artificial Sequence: Syntheticpolypeptide
MKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 66 - protein, 140 aa, synthetic construct
  REGION 1..140: note = Description of Artificial Sequence: Syntheticpolypeptide
MKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 67 - protein, 139 aa, synthetic construct
  REGION 1..139: note = Description of Artificial Sequence: Syntheticpolypeptide
MKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 68 - protein, 138 aa, synthetic construct
  REGION 1..138: note = Description of Artificial Sequence: Syntheticpolypeptide
MKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 69 - protein, 144 aa, synthetic construct
  REGION 1..144: note = Description of Artificial Sequence: Syntheticpolypeptide
ALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 70 - protein, 143 aa, synthetic construct
  REGION 1..143: note = Description of Artificial Sequence: Syntheticpolypeptide
ALTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 71 - protein, 142 aa, synthetic construct
  REGION 1..142: note = Description of Artificial Sequence: Syntheticpolypeptide
ALTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 72 - protein, 141 aa, synthetic construct
  REGION 1..141: note = Description of Artificial Sequence: Syntheticpolypeptide
ALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 73 - protein, 144 aa, synthetic construct; identical to SEQ ID NO: 20
  REGION 1..144: note = Description of Artificial Sequence: Syntheticpolypeptide

SEQ ID NO: 74 - protein, 143 aa, synthetic construct
  REGION 1..143: note = Description of Artificial Sequence: Syntheticpolypeptide
LTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 75 - protein, 142 aa, synthetic construct
  REGION 1..142: note = Description of Artificial Sequence: Syntheticpolypeptide
LTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 76 - protein, 141 aa, synthetic construct
  REGION 1..141: note = Description of Artificial Sequence: Syntheticpolypeptide
LTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 77 - protein, 140 aa, synthetic construct
  REGION 1..140: note = Description of Artificial Sequence: Syntheticpolypeptide
LTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 78 - protein, 143 aa, synthetic construct; identical to SEQ ID NO: 21
  REGION 1..143: note = Description of Artificial Sequence: Syntheticpolypeptide

SEQ ID NO: 79 - protein, 142 aa, synthetic construct
  REGION 1..142: note = Description of Artificial Sequence: Syntheticpolypeptide
TEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 80 - protein, 141 aa, synthetic construct
  REGION 1..141: note = Description of Artificial Sequence: Syntheticpolypeptide
TEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 81 - protein, 140 aa, synthetic construct
  REGION 1..140: note = Description of Artificial Sequence: Syntheticpolypeptide
TEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 82 - protein, 139 aa, synthetic construct
  REGION 1..139: note = Description of Artificial Sequence: Syntheticpolypeptide
TEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 83 - protein, 142 aa, synthetic construct; identical to SEQ ID NO: 22
  REGION 1..142: note = Description of Artificial Sequence: Syntheticpolypeptide

SEQ ID NO: 84 - protein, 141 aa, synthetic construct
  REGION 1..141: note = Description of Artificial Sequence: Syntheticpolypeptide
EKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 85 - protein, 140 aa, synthetic construct
  REGION 1..140: note = Description of Artificial Sequence: Syntheticpolypeptide
EKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 86 - protein, 139 aa, synthetic construct
  REGION 1..139: note = Description of Artificial Sequence: Syntheticpolypeptide
EKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 87 - protein, 138 aa, synthetic construct
  REGION 1..138: note = Description of Artificial Sequence: Syntheticpolypeptide
EKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 88 - protein, 141 aa, synthetic construct; identical to SEQ ID NO: 23
  REGION 1..141: note = Description of Artificial Sequence: Syntheticpolypeptide

SEQ ID NO: 89 - protein, 140 aa, synthetic construct
  REGION 1..140: note = Description of Artificial Sequence: Syntheticpolypeptide
KNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 90 - protein, 139 aa, synthetic construct
  REGION 1..139: note = Description of Artificial Sequence: Syntheticpolypeptide
KLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 91 - protein, 138 aa, synthetic construct
  REGION 1..138: note = Description of Artificial Sequence: Syntheticpolypeptide
KPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 92 - protein, 137 aa, synthetic construct
  REGION 1..137: note = Description of Artificial Sequence: Syntheticpolypeptide
KPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 93 - protein, 144 aa, synthetic construct
  REGION 1..144: note = Description of Artificial Sequence: Syntheticpolypeptide
ALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 94 - protein, 143 aa, synthetic construct
  REGION 1..143: note = Description of Artificial Sequence: Syntheticpolypeptide
ALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 95 - skipped

SEQ ID NO: 96 - protein, 142 aa, synthetic construct
  REGION 1..142: note = Description of Artificial Sequence: Syntheticpolypeptide
ALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 97 - protein, 141 aa, synthetic construct
  REGION 1..141: note = Description of Artificial Sequence: Syntheticpolypeptide
ALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 98 - protein, 144 aa, synthetic construct; identical to SEQ ID NO: 25
  REGION 1..144: note = Description of Artificial Sequence: Syntheticpolypeptide

SEQ ID NO: 99 - protein, 143 aa, synthetic construct
  REGION 1..143: note = Description of Artificial Sequence: Syntheticpolypeptide
LTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 100 - protein, 142 aa, synthetic construct
  REGION 1..142: note = Description of Artificial Sequence: Syntheticpolypeptide
LTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 101 - protein, 141 aa, synthetic construct
  REGION 1..141: note = Description of Artificial Sequence: Syntheticpolypeptide
LTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 102 - protein, 140 aa, synthetic construct
  REGION 1..140: note = Description of Artificial Sequence: Syntheticpolypeptide
LTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 103 - protein, 143 aa, synthetic construct; identical to SEQ ID NO: 26
  REGION 1..143: note = Description of Artificial Sequence: Syntheticpolypeptide

SEQ ID NO: 104 - protein, 142 aa, synthetic construct
  REGION 1..142: note = Description of Artificial Sequence: Syntheticpolypeptide
TEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 105 - protein, 141 aa, synthetic construct
  REGION 1..141: note = Description of Artificial Sequence: Syntheticpolypeptide
TEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 106 - protein, 140 aa, synthetic construct
  REGION 1..140: note = Description of Artificial Sequence: Syntheticpolypeptide
TEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 107 - protein, 139 aa, synthetic construct
  REGION 1..139: note = Description of Artificial Sequence: Syntheticpolypeptide
TEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 108 - protein, 142 aa, synthetic construct; identical to SEQ ID NO: 27
  REGION 1..142: note = Description of Artificial Sequence: Syntheticpolypeptide

SEQ ID NO: 109 - protein, 141 aa, synthetic construct
  REGION 1..141: note = Description of Artificial Sequence: Syntheticpolypeptide
EKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 110 - protein, 140 aa, synthetic construct
  REGION 1..140: note = Description of Artificial Sequence: Syntheticpolypeptide
EKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 111 - protein, 139 aa, synthetic construct
  REGION 1..139: note = Description of Artificial Sequence: Syntheticpolypeptide
EKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 112 - protein, 138 aa, synthetic construct
  REGION 1..138: note = Description of Artificial Sequence: Syntheticpolypeptide
EKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 113 - protein, 141 aa, synthetic construct; identical to SEQ ID NO: 28
  REGION 1..141: note = Description of Artificial Sequence: Syntheticpolypeptide

SEQ ID NO: 114 - protein, 140 aa, synthetic construct
  REGION 1..140: note = Description of Artificial Sequence: Syntheticpolypeptide
KNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 115 - protein, 139 aa, synthetic construct
  REGION 1..139: note = Description of Artificial Sequence: Syntheticpolypeptide
KLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 116 - protein, 138 aa, synthetic construct
  REGION 1..138: note = Description of Artificial Sequence: Syntheticpolypeptide
KPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 117 - protein, 137 aa, synthetic construct
  REGION 1..137: note = Description of Artificial Sequence: Syntheticpolypeptide
KPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 118 - protein, 146 aa, synthetic construct; identical to SEQ ID NO: 14
  REGION 1..146: note = Description of Artificial Sequence: Syntheticpolypeptide

SEQ ID NO: 119 - protein, 144 aa, synthetic construct; identical to SEQ ID NO: 46
  REGION 1..144: note = Description of Artificial Sequence: Syntheticpolypeptide

SEQ ID NO: 120 - protein, 143 aa, synthetic construct
  REGION 1..143: note = Description of Artificial Sequence: Syntheticpolypeptide
MALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 121 - protein, 142 aa, synthetic construct
  REGION 1..142: note = Description of Artificial Sequence: Syntheticpolypeptide
MALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 122 - protein, 145 aa, synthetic construct; identical to SEQ ID NO: 15
  REGION 1..145: note = Description of Artificial Sequence: Syntheticpolypeptide

SEQ ID NO: 123 - protein, 144 aa, synthetic construct
  REGION 1..144: note = Description of Artificial Sequence: Syntheticpolypeptide
MLTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 124 - protein, 143 aa, synthetic construct
  REGION 1..143: note = Description of Artificial Sequence: Syntheticpolypeptide
MLTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 125 - protein, 142 aa, synthetic construct
  REGION 1..142: note = Description of Artificial Sequence: Syntheticpolypeptide
MLTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 126 - protein, 141 aa, synthetic construct
  REGION 1..141: note = Description of Artificial Sequence: Syntheticpolypeptide
MLTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 127 - protein, 144 aa, synthetic construct; identical to SEQ ID NO: 16
  REGION 1..144: note = Description of Artificial Sequence: Syntheticpolypeptide

SEQ ID NO: 128 - protein, 143 aa, synthetic construct
  REGION 1..143: note = Description of Artificial Sequence: Syntheticpolypeptide
MTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 129 - protein, 142 aa, synthetic construct
  REGION 1..142: note = Description of Artificial Sequence: Syntheticpolypeptide
MTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 130 - protein, 141 aa, synthetic construct
  REGION 1..141: note = Description of Artificial Sequence: Syntheticpolypeptide
MTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 131 - protein, 140 aa, synthetic construct
  REGION 1..140: note = Description of Artificial Sequence: Syntheticpolypeptide
MTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 132 - protein, 143 aa, synthetic construct; identical to SEQ ID NO: 17
  REGION 1..143: note = Description of Artificial Sequence: Syntheticpolypeptide

SEQ ID NO: 133 - protein, 142 aa, synthetic construct
  REGION 1..142: note = Description of Artificial Sequence: Syntheticpolypeptide
MEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 134 - protein, 141 aa, synthetic construct
  REGION 1..141: note = Description of Artificial Sequence: Syntheticpolypeptide
MEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 135 - protein, 140 aa, synthetic construct
  REGION 1..140: note = Description of Artificial Sequence: Syntheticpolypeptide
MEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 136 - protein, 139 aa, synthetic construct
  REGION 1..139: note = Description of Artificial Sequence: Syntheticpolypeptide
MEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 137 - protein, 142 aa, synthetic construct; identical to SEQ ID NO: 18
  REGION 1..142: note = Description of Artificial Sequence: Syntheticpolypeptide

SEQ ID NO: 138 - protein, 141 aa, synthetic construct
  REGION 1..141: note = Description of Artificial Sequence: Syntheticpolypeptide
MKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 139 - protein, 140 aa, synthetic construct
  REGION 1..140: note = Description of Artificial Sequence: Syntheticpolypeptide
MKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 140 - protein, 139 aa, synthetic construct
  REGION 1..139: note = Description of Artificial Sequence: Syntheticpolypeptide
MKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 141 - protein, 138 aa, synthetic construct
  REGION 1..138: note = Description of Artificial Sequence: Syntheticpolypeptide
MKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 142 - protein, 140 aa, synthetic construct
  REGION 1..140: note = Description of Artificial Sequence: Syntheticpolypeptide
  MOD_RES 67: note = Any amino acid
FNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 143 - protein, 139 aa, synthetic construct
  REGION 1..139: note = Description of Artificial Sequence: Syntheticpolypeptide
  MOD_RES 66: note = Any amino acid
NLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 144 - protein, 137 aa, synthetic construct
  REGION 1..137: note = Description of Artificial Sequence: Syntheticpolypeptide
  MOD_RES 64: note = Any amino acid
PPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 145 - protein, 136 aa, synthetic construct
  REGION 1..136: note = Description of Artificial Sequence: Syntheticpolypeptide
  MOD_RES 63: note = Any amino acid
PGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 146 - protein, 140 aa, synthetic construct
  REGION 1..140: note = Description of Artificial Sequence: Syntheticpolypeptide
  MOD_RES 67: note = Any amino acid
FNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 147 - protein, 139 aa, synthetic construct
  REGION 1..139: note = Description of Artificial Sequence: Syntheticpolypeptide
  MOD_RES 66: note = Any amino acid
NLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 148 - protein, 137 aa, synthetic construct
  REGION 1..137: note = Description of Artificial Sequence: Syntheticpolypeptide
  MOD_RES 64: note = Any amino acid
PPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 149 - protein, 136 aa, synthetic construct
  REGION 1..136: note = Description of Artificial Sequence: Syntheticpolypeptide
  MOD_RES 63: note = Any amino acid
PGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 150 - protein, 144 aa, synthetic construct
  REGION 1..144: note = Description of Artificial Sequence: Syntheticpolypeptide
  MOD_RES 71: note = Any amino acid
ALTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 151 - protein, 143 aa, synthetic construct
  REGION 1..143: note = Description of Artificial Sequence: Syntheticpolypeptide
  MOD_RES 70: note = Any amino acid
ALTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 152 - protein, 143 aa, synthetic construct
  REGION 1..143: note = Description of Artificial Sequence: Syntheticpolypeptide
  MOD_RES 70: note = Any amino acid
ALTEKPPGGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 153 - protein, 141 aa, synthetic construct
  REGION 1..141: note = Description of Artificial Sequence: Syntheticpolypeptide
  MOD_RES 68: note = Any amino acid
ALTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 154 - protein, 144 aa, synthetic construct
  REGION 1..144: note = Description of Artificial Sequence: Syntheticpolypeptide
  MOD_RES 71: note = Any amino acid
LTEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 155 - protein, 143 aa, synthetic construct
  REGION 1..143: note = Description of Artificial Sequence: Syntheticpolypeptide
  MOD_RES 70: note = Any amino acid
LTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 156 - protein, 142 aa, synthetic construct
  REGION 1..142: note = Description of Artificial Sequence: Syntheticpolypeptide
  MOD_RES 69: note = Any amino acid
LTEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 157 - protein, 141 aa, synthetic construct
  REGION 1..141: note = Description of Artificial Sequence: Syntheticpolypeptide
  MOD_RES 68: note = Any amino acid
LTEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 158 - protein, 140 aa, synthetic construct
  REGION 1..140: note = Description of Artificial Sequence: Syntheticpolypeptide
  MOD_RES 67: note = Any amino acid
LTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 159 - protein, 143 aa, synthetic construct
  REGION 1..143: note = Description of Artificial Sequence: Syntheticpolypeptide
  MOD_RES 70: note = Any amino acid
TEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 160 - protein, 142 aa, synthetic construct
  REGION 1..142: note = Description of Artificial Sequence: Syntheticpolypeptide
  MOD_RES 69: note = Any amino acid
TEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 161 - protein, 141 aa, synthetic construct
  REGION 1..141: note = Description of Artificial Sequence: Syntheticpolypeptide
  MOD_RES 68: note = Any amino acid
TEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 162 - protein, 140 aa, synthetic construct
  REGION 1..140: note = Description of Artificial Sequence: Syntheticpolypeptide
  MOD_RES 67: note = Any amino acid
TEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 163 - protein, 139 aa, synthetic construct
  REGION 1..139: note = Description of Artificial Sequence: Syntheticpolypeptide
  MOD_RES 66: note = Any amino acid
TEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 164 - protein, 142 aa, synthetic construct
  REGION 1..142: note = Description of Artificial Sequence: Syntheticpolypeptide
  MOD_RES 69: note = Any amino acid
EKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 165 - protein, 141 aa, synthetic construct
  REGION 1..141: note = Description of Artificial Sequence: Syntheticpolypeptide
  MOD_RES 68: note = Any amino acid
EKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 166 - protein, 140 aa, synthetic construct
  REGION 1..140: note = Description of Artificial Sequence: Syntheticpolypeptide
  MOD_RES 67: note = Any amino acid
EKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 167 - protein, 139 aa, synthetic construct
  REGION 1..139: note = Description of Artificial Sequence: Syntheticpolypeptide
  MOD_RES 66: note = Any amino acid
EKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 168 - protein, 138 aa, synthetic construct
  REGION 1..138: note = Description of Artificial Sequence: Syntheticpolypeptide
  MOD_RES 65: note = Any amino acid
EKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 169 - protein, 141 aa, synthetic construct
  REGION 1..141: note = Description of Artificial Sequence: Syntheticpolypeptide
  MOD_RES 68: note = Any amino acid
KFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 170 - protein, 140 aa, synthetic construct
  REGION 1..140: note = Description of Artificial Sequence: Syntheticpolypeptide
  MOD_RES 67: note = Any amino acid
KNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 171 - protein, 139 aa, synthetic construct
  REGION 1..139: note = Description of Artificial Sequence: Syntheticpolypeptide
  MOD_RES 66: note = Any amino acid
KLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 172 - protein, 138 aa, synthetic construct
  REGION 1..138: note = Description of Artificial Sequence: Syntheticpolypeptide
  MOD_RES 65: note = Any amino acid
KPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 173 - protein, 137 aa, synthetic construct
  REGION 1..137: note = Description of Artificial Sequence: Syntheticpolypeptide
  MOD_RES 64: note = Any amino acid
KPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 174 - protein, 140 aa, synthetic construct; identical to SEQ ID NO: 146
  REGION 1..140: note = Description of Artificial Sequence: Syntheticpolypeptide
  MOD_RES 67: note = Any amino acid

SEQ ID NO: 175 - protein, 145 aa, synthetic construct
  REGION 1..145: note = Description of Artificial Sequence: Syntheticpolypeptide
  MOD_RES 72: note = Any amino acid
ALTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 176 - protein, 144 aa, synthetic construct
  REGION 1..144: note = Description of Artificial Sequence: Syntheticpolypeptide
  MOD_RES 71: note = Any amino acid
LTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 177 - protein, 143 aa, synthetic construct
  REGION 1..143: note = Description of Artificial Sequence: Syntheticpolypeptide
  MOD_RES 70: note = Any amino acid
TEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 178 - protein, 142 aa, synthetic construct
  REGION 1..142: note = Description of Artificial Sequence: Syntheticpolypeptide
  MOD_RES 69: note = Any amino acid
EKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 179 - protein, 141 aa, synthetic construct
  REGION 1..141: note = Description of Artificial Sequence: Syntheticpolypeptide
  MOD_RES 68: note = Any amino acid
KFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 180 - protein, 141 aa, synthetic construct; identical to SEQ ID NO: 179
  REGION 1..141: note = Description of Artificial Sequence: Syntheticpolypeptide
  MOD_RES 68: note = Any amino acid

SEQ ID NO: 181 - protein, 144 aa, synthetic construct
  REGION 1..144: note = Description of Artificial Sequence: Syntheticpolypeptide
  MOD_RES 71: note = Any amino acid
ALTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 182 - protein, 143 aa, synthetic construct; identical to SEQ ID NO: 151
  REGION 1..143: note = Description of Artificial Sequence: Syntheticpolypeptide
  MOD_RES 70: note = Any amino acid

SEQ ID NO: 183 - protein, 142 aa, synthetic construct
  REGION 1..142: note = Description of Artificial Sequence: Syntheticpolypeptide
  MOD_RES 69: note = Any amino acid
ALTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 184 - protein, 141 aa, synthetic construct
  REGION 1..141: note = Description of Artificial Sequence: Syntheticpolypeptide
  MOD_RES 68: note = Any amino acid
ALTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 185 - protein, 144 aa, synthetic construct; identical to SEQ ID NO: 176
  REGION 1..144: note = Description of Artificial Sequence: Syntheticpolypeptide
  MOD_RES 71: note = Any amino acid

SEQ ID NO: 186 - protein, 143 aa, synthetic construct
  REGION 1..143: note = Description of Artificial Sequence: Syntheticpolypeptide
  MOD_RES 70: note = Any amino acid
LTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 187 - protein, 142 aa, synthetic construct
  REGION 1..142: note = Description of Artificial Sequence: Syntheticpolypeptide
  MOD_RES 69: note = Any amino acid
LTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 188 - protein, 141 aa, synthetic construct
  REGION 1..141: note = Description of Artificial Sequence: Syntheticpolypeptide
  MOD_RES 68: note = Any amino acid
LTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 189 - protein, 140 aa, synthetic construct
  REGION 1..140: note = Description of Artificial Sequence: Syntheticpolypeptide
  MOD_RES 67: note = Any amino acid
LTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 190 - protein, 143 aa, synthetic construct; identical to SEQ ID NO: 177
  REGION 1..143: note = Description of Artificial Sequence: Syntheticpolypeptide
  MOD_RES 70: note = Any amino acid

SEQ ID NO: 191 - protein, 142 aa, synthetic construct
  REGION 1..142: note = Description of Artificial Sequence: Syntheticpolypeptide
  MOD_RES 69: note = Any amino acid
TEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 192 - protein, 141 aa, synthetic construct
  REGION 1..141: note = Description of Artificial Sequence: Syntheticpolypeptide
  MOD_RES 68: note = Any amino acid
TEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 193 - protein, 140 aa, synthetic construct
  REGION 1..140: note = Description of Artificial Sequence: Syntheticpolypeptide
  MOD_RES 67: note = Any amino acid
TEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 194 - protein, 139 aa, synthetic construct
  REGION 1..139: note = Description of Artificial Sequence: Syntheticpolypeptide
  MOD_RES 66: note = Any amino acid
TEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 195 - protein, 142 aa, synthetic construct; identical to SEQ ID NO: 178
  REGION 1..142: note = Description of Artificial Sequence: Syntheticpolypeptide
  MOD_RES 69: note = Any amino acid

SEQ ID NO: 196 - protein, 141 aa, synthetic construct
  REGION 1..141: note = Description of Artificial Sequence: Syntheticpolypeptide
  MOD_RES 68: note = Any amino acid
EKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 197 - protein, 140 aa, synthetic construct
  REGION 1..140: note = Description of Artificial Sequence: Syntheticpolypeptide
  MOD_RES 67: note = Any amino acid
EKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 198 - protein, 139 aa, synthetic construct
  REGION 1..139: note = Description of Artificial Sequence: Syntheticpolypeptide
  MOD_RES 66: note = Any amino acid
EKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 199 - protein, 138 aa, synthetic construct
  REGION 1..138: note = Description of Artificial Sequence: Syntheticpolypeptide
  MOD_RES 65: note = Any amino acid
EKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 200 - protein, 141 aa, synthetic construct; identical to SEQ ID NO: 179
  REGION 1..141: note = Description of Artificial Sequence: Syntheticpolypeptide
  MOD_RES 68: note = Any amino acid

SEQ ID NO: 201 - protein, 140 aa, synthetic construct
  REGION 1..140: note = Description of Artificial Sequence: Syntheticpolypeptide
  MOD_RES 67: note = Any amino acid
KNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 202 - protein, 139 aa, synthetic construct
  REGION 1..139: note = Description of Artificial Sequence: Syntheticpolypeptide
  MOD_RES 66: note = Any amino acid
KLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 203 - protein, 138 aa, synthetic construct
  REGION 1..138: note = Description of Artificial Sequence: Syntheticpolypeptide
  MOD_RES 65: note = Any amino acid
KPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 204 - protein, 137 aa, synthetic construct
  REGION 1..137: note = Description of Artificial Sequence: Syntheticpolypeptide
  MOD_RES 64: note = Any amino acid
KPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 205 - protein, 140 aa, synthetic construct
  REGION 1..140: note = Description of Artificial Sequence: Syntheticpolypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 206 - protein, 140 aa, synthetic construct
  REGION 1..140: note = Description of Artificial Sequence: Syntheticpolypeptide
FNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 207 - protein, 145 aa, synthetic construct
  REGION 1..145: note = Description of Artificial Sequence: Syntheticpolypeptide
MALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

(19) United States
(12) Patent Application Publication
(10) Pub. No.: US 2025/0066440 A1
(43) Pub. Date: Feb. 27, 2025

(54) METHODS OF TREATMENT USING MODIFIED FGF-1 POLYPEPTIDES

(71) Applicant: Trefoil Therapeutics, Inc., San Diego, CA (US)

(72) Inventor: David EVELETH, San Diego, CA (US)

(21) Appl. No.: 18/602,496

(22) Filed: Mar. 12, 2024

Related U.S. Application Data
(63) Continuation of application No. PCT/US2022/043378, filed on Sep. 13, 2022.
(60) Provisional application No. 63/243,898, filed on Sep. 14, 2021.

(51) Int. Cl. C07K 14/50; A61K 9/00; A61K 38/00; A61K 47/26; A61P 27/04

(52) U.S. Cl. CPC C07K 14/50; A61K 9/0048; A61K 47/26; A61P 27/04; A61K 38/00

(57) ABSTRACT

Described herein are methods of treating a disease, disorder or a condition associated with dry eye caused by a multifactorial disease of ocular surface, comprising administering to a subject in need thereof a pharmaceutical composition comprising a therapeutically effective amount of a modified FGF-1 polypeptide.

### CROSS REFERENCE

This application is a continuation of International Patent Application No. PCT/US2022/043378, filed Sep. 13, 2022, which claims benefit of U.S. Provisional Patent Application No. 63/243,898, filed Sep. 14, 2021, which is incorporated by reference herein in its entirety.

### REFERENCE TO SEQUENCE LISTING SUBMITTED ELECTRONICALLY

This application incorporates by reference a Sequence Listing submitted with this application as a text file entitled “45341-712_601_SL.xml” created on Aug. 30, 2022 and having a size of 311,900 bytes.

### BACKGROUND

FGFs are large polypeptides widely expressed in developing and adult tissues (Baird et al., Cancer Cells, 3:239-243, 1991) and play roles in multiple physiological functions (McKeehan et al., Prog. Nucleic Acid Res. Mol. Biol. 59:135-176, 1998; Burgess, W. H. et al., Annu Rev. Biochem. 58:575-606 (1989). The FGF family includes at least twenty-two members (Reuss et al., Cell Tissue Res. 313:139-157 (2003)).

### SUMMARY

Provided herein in one embodiment is a method of treating dry eye, the method comprising: administering to a subject in need thereof a pharmaceutical composition comprising a therapeutically effective amount of a modified FGF-1 polypeptide, wherein the modified FGF-1 polypeptide comprises a sequence comprising the mutations Cys16Ser, Ala66Cys, and Cys117Val relative to the wild-type FGF-1 sequence of SEQ ID NO: 1. In some embodiments, meibomian gland dysfunction, lacrimal gland insufficiency, lacrimal duct obstruction, reflex hyposecretion, Sjogren's syndrome, eyelid aperture disorder, blink disorder, or ocular surface disorders. In some embodiments, the dye eye is caused by the Sjogren's syndrome and the Sjogren's syndrome is a primary Sjogren's syndrome or a secondary Sjogren's syndrome. In some embodiments, the dry eye is caused by an autoimmune disease. In some embodiments, the autoimmune disease is at least one of: systemic lupus erythematosus, scleroderma, or rheumatoid arthritis. In some embodiments, the dry eye is caused by a disease selected from the group consisting of: GVHD-induced dry eye, radiation-induced dry eye, keratoconjunctivitis sicca, Stevens-Johnson syndrome, lacrimo-auriculo-dento-digital syndrome, ocular cicatrical pemphigoid, ocular surface infection, Riley-Day syndrome, congenital alacrima, nutritional disorders or deficiencies, a glandular destruction, a tissue destruction, an immunodeficient disorder, an inability to blink in comatose patient, a systemic disease, a virus infection, a bacterial infection, a skin disease, an environmental exposure to airborne particulates, smoke, or smog, contact lens intolerance, and a long-term exposure to display devices. In some embodiments, the dry eye is caused by the systemic disease, wherein the systemic disease comprises at least one of Parkinson's disease, diabetes, or thyroid disease. In some embodiments, the dry eye is caused by the virus infection, wherein the virus comprises at least one of human T-cell lymphotropic virus (HTLV), human immunodeficiency virus (HIV), Epstein-Barr virus (EBV), hepatitis C virus (HCV), Influenza, or Measles virus. In some embodiments, the dry eye is caused by the skin disease, wherein the skin disease comprises at least one of: rosacea, blepharitis, or pruritus. In some embodiments, the pharmaceutical composition comprises at least about 5% concentration of a humectant. In some embodiments, the humectant is selected from the group consisting of Glycerin, Maltitol, Propylene Glycol, Sorbitol, and Triacetin. In some embodiments, the pharmaceutical composition comprises at least about 0.1% of a surfactant. In some embodiments, the surfactant comprises polysorbate 80 or polysorbate 20. In some embodiments, the pharmaceutical composition comprises about 1 mM citrate or Histidine, about 5% sorbitol, about 0.1% polysorbate 80, and has a pH of about 5.8. In some embodiments, the pharmaceutical composition comprises about 20 mM phosphate buffered saline, about 0.1% polysorbate 80, and has a pH of about 7.4.

Provided herein in one embodiment is a method of treating keratoconjunctivitis sicca, the method comprising: administering to a subject in need thereof a pharmaceutical composition comprising a therapeutically effective amount of a modified FGF-1 polypeptide, wherein the modified FGF-1 polypeptide comprises a sequence comprising the mutations Cys16Ser, Ala66Cys, and Cys117Val relative to the wild-type FGF-1 sequence of SEQ ID NO: 1, wherein the pharmaceutical composition comprises about 10 mM citrate or Histidine, about 5% sorbitol, about 0.1% polysorbate 80, and has a pH of about 5.8. In some embodiments, a method of treating keratoconjunctivitis sicca, the method comprising: administering to a subject in need thereof a pharmaceutical composition comprising a therapeutically effective amount of a modified FGF-1 polypeptide, wherein the modified FGF-1 polypeptide comprises a sequence comprising the mutations Cys16Ser, Ala66Cys, and Cys17Val relative to the wild-type FGF-1 sequence of SEQ ID NO: 1, wherein the pharmaceutical composition comprises about 20 mM phosphate buffered saline, about 0.1% polysorbate 80, and has a pH of about 7.4.

In some embodiments, the modified FGF-1 polypeptide comprises an N-terminal methionine upstream of the first residue of SEQ ID NO: 1. In some embodiments, the modified FGF-1 polypeptide comprises a sequence that has at least about 80% sequence identity to SEQ ID NO: 2. In some embodiments, the modified FGF-1 polypeptide comprises a sequence that has at least about 90% sequence identity to SEQ ID NO: 2. In some embodiments, the modified FGF-1 polypeptide comprises a sequence that has at least about 95% sequence identity to SEQ ID NO: 2. In some embodiments, the modified FGF-1 polypeptide comprises a sequence that has at least about 98% sequence identity to SEQ ID NO: 2. In some embodiments, the modified FGF-1 polypeptide comprises a sequence that has at least about 80% sequence identity to SEQ ID NO: 205. In some embodiments, the modified FGF-1 polypeptide comprises a sequence that has at least about 90% i sequence identity to SEQ ID NO: 205. In some embodiments, the modified FGF-1 polypeptide comprises a sequence that has at least about 95% sequence identity to SEQ ID NO: 205. In some embodiments, the modified FGF-1 polypeptide comprises a sequence that has at least about 98% sequence identity to SEQ ID NO: 205.

In some embodiments, the subject is human. In some embodiments, administering the therapeutically effective amount of a pharmaceutical composition comprising a modified FGF-1 polypeptide reduces or eliminates a clinical symptom associated with dry eye. In some embodiments, the clinical symptom comprises at least one of: ocular dryness, grittiness, irritation, mucoid discharge, hyperemia, photophobia, or blurry vision. In some embodiments, the pharmaceutical composition comprises a formulation for ocular delivery. In some embodiments, the pharmaceutical composition comprises an injectable formulation for ocular delivery. In some embodiments, the injectable formulation is administered via intracameral or intravitreal injection. In some embodiments, the formulation is administered by topical application. In some embodiments, the formulation is administered as an eye drop.

### INCORPORATION BY REFERENCE

All publications and patent applications mentioned in this specification are herein incorporated by reference in their entireties to the same extent as if each individual publication or patent application was specifically and individually indicated to be incorporated by reference.

### DETAILED DESCRIPTION

Dry eye has been defined as “a multifactorial disease of ocular surface characterized by a loss of homeostasis of the tear film, and accompanied by ocular symptoms, in which tear film instability and hyperosmolarity, ocular surface inflammation and damage, and neurosensory abnormalities have etiologic roles” at the Dry Eye Workshop (DEWS II, August, 2017). Dry eye is a prevalent ocular disorder characterized by bilateral reduced aqueous tear production and tear film instability. It affects around 5% to 40% of adults older than 40 years and the prevalence of dry eye is higher in women than men. Based on a recent study, more than 20 million people were estimated to have dry eye in the United States. Dry eye causes eye irritation, hyperemia, glare, eye fatigue, and blurred vision. Vision impairment in dry eye is due to the increased higher-order aberrations, or superficial punctate keratitis, and in severe cases, such as graft-versus-host disease (GVHD) or Stevens-Johnson syndrome (SJS), blindness from corneal opacification or ulceration may result.

Broadly, dry eye can be any condition or symptom associated with tear film instability and dysfunction (such as increased tear evaporation and/or reduced aqueous secretion). Among the indications that are referred to by the general term “dry eye” include but are not limited to: Keratoconjunctivitis sicca (KCS), meibomian gland dysfunction, age-related dry eye, Stevens-Johnson syndrome, Sjogren's syndrome, GVHD-induced dry eye, radiation-induced dry eye, lacrimo-auriculo-dento-digital syndrome, ocular cicatrical pemphigoid, corneal injury, ocular surface infection, Riley-Day syndrome, congenital alacrima, nutritional disorders or deficiencies (including vitamin deficiencies), pharmacologic side effects, glandular and tissue destruction, autoimmune and other immunodeficient disorders, and inability to blink in comatose patients. Also included are dry eye symptoms caused by environmental exposure to airborne particulates, smoke, smog, and excessively dry air, contact lens intolerance and eye stress caused by computer work or computer gaming. These conditions affect the quality and stability of the tear film, which results in dry eye signs and symptoms. Laser assisted vision correction procedures such as photorefractive keratectomy (PRK), laser-assisted sub-epithelial keratectomy (LASEK) and laser-assisted in situ keratomileusis (LASIK) also negatively influence tear film functionality and frequently cause (temporary) dry eye signs and symptoms.

There are largely two major types of dry eye: aqueous-deficient dry eye due to lacrimal gland diseases and evaporative dry eye which is mainly due to meibomian gland diseases. Within the class of aqueous-deficient forms of dry eye, two major subtypes are differentiated, Sjogren and non-Sjogren. Sjogren syndrome patients typically suffer from autoimmune disorders in which the lacrimal glands are invaded by activated T-cells, which leads not only to keratoconjunctivitis sicca but also to a dry mouth condition. The Sjogren syndrome can be a primary disease or result from other autoimmune diseases such as systemic lupus erythrematosus or rheumatoid arthritis. Non-Sjogren patients suffering from an aqueous-deficient dry eye usually have a lacrimal gland insufficiency, lacrimal duct obstruction or reflex hyposecretion. The second major class, evaporative dry eye, is also somewhat heterogeneous and can develop as a result of diverse root causes. One of the major causes is meibomian gland disease, eyelid aperture disorders, blink disorders (as in Parkinson disease) or ocular surface disorders (as in allergic conjunctivitis).

Although it seems that dry eyes can occur as a result of a number of unrelated pathogenic causes, all manifestations of the complication have a common effect, namely a violation of the tear film of the eye, which leads to dehydration of the external surface exposed to the external effects and many of the symptoms described herein. Despite the high incidence of dry eye disease, there is currently no consistently effective treatment for this condition and it still remains a therapeutic challenge. As such, there is a need for new therapeutic modalities to treat dry eye.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer softwares such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Alignment for purposes of determining percent amino acid sequence identity can for example be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration No. TXU510087. The ALIGN-2 program can be compiled for use on a UNIX operating system, such as a digital UNIX V4.0D. All sequence comparison parameters are set by the ALIGN-2 program and do not vary.

Definition of standard chemistry terms may be found in reference works, including but not limited to, Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “treat,” “treating” or “treatment” include alleviating, abating or ameliorating a disease, disorder or condition symptoms, preventing additional symptoms, ameliorating or preventing the underlying metabolic causes of symptoms, inhibiting the disease, disorder, or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition. The terms “treat,” “treating” or “treatment”, include, but are not limited to, prophylactic and/or therapeutic treatments.

The term “acceptable” or “pharmaceutically acceptable”, with respect to a formulation, composition or ingredient, refers to having no persistent detrimental effect on the general health of the subject being treated or does not abrogate the biological activity or properties of the modified FGF described herein, and is relatively nontoxic.

The term “amelioration” of the symptoms of a particular disease, disorder or condition by administration of a particular modified FGF or pharmaceutical composition refers to any lessening of severity, delay in onset, slowing of progression, or shortening of duration, whether permanent or temporary, lasting or transient that can be attributed to or associated with administration of the modified FGF or pharmaceutical composition.

The term “combination” or “pharmaceutical combination” as used herein, means a product that results from the mixing or combining of more than one active ingredient and includes both fixed and non-fixed combinations of the active ingredients. The term “fixed combination” means that one active ingredient (e.g., a modified FGF-1 polypeptide) and a co-agent are both administered to a patient simultaneously in the form of a single entity or dosage. The term “non-fixed combination” means that one active ingredient (e.g., a modified FGF-1 polypeptide) and a co-agent are administered to a patient as separate entities either simultaneously, concurrently or sequentially with no specific intervening time limits, wherein such administration provides effective levels of the two agents in the body of the patient. The latter also applies to cocktail therapy, e.g., the administration of three or more active ingredients.

The term “pharmaceutical composition” as used herein refers to one or more modified FGF (e.g., FGF-1) polypeptides with one or more other chemical components, such as carriers, stabilizers, diluents, dispersing agents, suspending agents, thickening agents, and/or excipients. The pharmaceutical composition facilitates administration of the modified FGF-1 polypeptides to an organism. Multiple techniques of administering a modified FGF-1 polypeptide exist in the art including, but not limited to: topical, ophthalmic, intraocular, periocular, intravenous, oral, aerosol, and parenteral administration.

The term “carrier,” as used herein, refers to relatively nontoxic chemical compounds or agents that facilitate the incorporation of an agent of interest (e.g., a modified FGF) into cells or tissues.

The term “diluent” refers to chemical compounds that are used to dilute the agent of interest (e.g., a modified FGF polypeptide such as a modified FGF-1 polypeptide) prior to delivery. Diluents can also be used to stabilize agents because they can provide a more stable environment. Salts dissolved in buffered solutions (which also can provide pH control or maintenance) are utilized as diluents in the art, including, but not limited to a phosphate buffered saline solution.

The terms “co-administration” or the like, are meant to encompass administration of the selected agents (e.g., a modified FGF-1 polypeptide or composition thereof and a co-agent) to a single patient, and are intended to include treatment regimens in which the agents are administered by the same or different route of administration or at the same or different time.

The terms “effective amount” or “therapeutically effective amount,” refer to a sufficient amount of a modified FGF-1 polypeptide, agent, combination or pharmaceutical composition described herein administered which will relieve to some extent one or more of the symptoms of the disease, disorder or condition being treated. The result can be reduction and/or alleviation of the signs, symptoms, or causes of a disease, or any other desired alteration of a biological system. For example, an “effective amount” for therapeutic uses is the amount of the modified FGF-1 polypeptide, agent, combination or pharmaceutical composition required to provide a desired pharmacologic effect, therapeutic improvement, or clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. It is understood that “an effect amount” can vary from subject to subject due to variation in metabolism of the modified FGF polypeptide such as a modified FGF-1 polypeptide, combination, or pharmaceutical composition, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “prophylactically effective amount,” refers that amount of a modified FGF-1 polypeptide, compound, agent, combination or pharmaceutical composition described herein applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” or “patient” as used herein, refers to an animal, which is the object of treatment, observation or experiment. By way of example only, a subject may be, but is not limited to, a mammal including, but not limited to, a human.

The terms “enhance” or “enhancing” means to increase or prolong either in potency or duration a desired effect. By way of example, “enhancing” the effect of therapeutic agents singly or in combination refers to the ability to increase or prolong, either in potency, duration and/or magnitude, the effect of the agents on the treatment of a disease, disorder or condition. When used in a patient, amounts effective for this use will depend on the severity and course of the disease, disorder or condition, previous therapy, the patient's health status and response to the drugs, and the judgment of the treating physician.

The term “modulate,” means to interact with a target (e.g., a FGF receptor) either directly or indirectly so as to alter the activity of the target, including, by way of example only, to enhance the activity of the target, to inhibit or antagonize the activity of the target, to limit the activity of the target, or to extend the activity of the target. In some embodiments, modified FGF-1 polypeptides and pharmaceutical compositions described herein can modulate the activity of one or more respective targets (e.g., one or more FGF receptors). In some embodiments, the modified FGF-1 polypeptides described herein modulate (e.g., increase) the activity of one or more FGF receptors on a cell (e.g., a corneal endothelial cell), resulting, e.g., in cell migration and/or cell proliferation.

As used herein, the term “target” or refers to a biological molecule (e.g., a target protein or protein complex), such as an FGF receptor, or a portion of a biological molecule capable of being bound by a selective binding agent (e.g., a modified FGF) or pharmaceutical composition described herein. As used herein, the term “non-target” refers to a biological molecule or a portion of a biological molecule that is not selectively bound by a selective binding agent or pharmaceutical composition described herein.

The term “target activity” or “cell response” refers to a biological activity capable of being modulated by a modified FGF polypeptide such as a modified FGF-1 polypeptide or any cellular response that results from the binding of a modified FGF-1 polypeptide to a FGF receptor. Certain exemplary target activities and cell responses include, but are not limited to, binding affinity, signal transduction, gene expression, cell migration, cell proliferation, cell differentiation, and amelioration of one or more symptoms associated with an ocular disease, disorder or condition.

### Modified FGF-1 Polypeptides for Treating Dry Eye

FGFs stimulate a family of seven FGF receptor isoforms, and each FGF stimulates a different pattern of receptors to achieve its specific effect. See, e.g., Ornitz et al. (1996) The Journal of biological chemistry, 1996, 271(25):15292-7; Zhang et al. (2006) The Journal of biological chemistry, 2006, 281(23):15694-700). In some embodiments, a modified FGF-1 polypeptide is preferable because it binds to and stimulates all seven FGF receptor isoforms. See Ornitz et al. (1996) The Journal of biological chemistry, 1996, 271(25):15292-7.

Certain embodiments disclosed herein relate to methods of treating dry eye in a subject in need thereof comprising administering a modified FGF (e.g., FGF-1) polypeptide or a pharmaceutical composition (e.g., an ophthalmic formulation) comprising a modified FGF described herein (e.g., FGF-1 polypeptide). In some embodiments, the methods comprise treating dry eye by administering a modified FGF (e.g., FGF-1) polypeptide described herein or a pharmaceutical composition (e.g., an ophthalmic formulation) comprising the modified FGF (e.g., FGF-1) polypeptide. A modified FGF-polypeptide, as used herein, refers to a recombinant FGF that includes a substitution or mutation of one or more different amino acid residues and/or one or more deletions of one or more amino acid residues and/or one or more additions of one or more amino acid residues of a wild-type FGF-polypeptide (e.g., a wild-type FGF-1 polypeptide of SEQ ID NO: 1). In some embodiments, the modified FGF-polypeptide comprises a modified FGF-1 polypeptide with a substitution or mutation of one or more different amino acid residues and/or one or more deletions of one or more amino acid residues and/or one or more additions of one or more amino acid residues relative to the amino acid sequence of SEQ ID NO: 1.

Provided herein, in some embodiments, are methods of treating dry eye comprising administering to the subject a modified FGF-1 polypeptide comprising the sequence set forth as SEQ ID NO: 1, with one or more mutations (e.g., substitution), additions or deletions, wherein the modified polypeptide further comprises a methionine residue upstream to the first residue of SEQ ID NO: 1. In some embodiments, the modified FGF-1 polypeptide comprising the N-terminal methionine (N-Met) residue is a mature form of the FGF-1 polypeptide. In some embodiments, the modified FGF-1 polypeptide comprises one or more mutations at positions 12, 16, 66, 117, and 134 of SEQ ID NO: 1. In some embodiments, the modified FGF-1 polypeptide is expressed in a host cell with a methionine residue upstream to the first residue of SEQ ID NO: 1. In some embodiments, the modified FGF-1 polypeptide is not subject to N-terminal processing for removal of the N-Met residue during polypeptide maturation. In some embodiments, the mature form of a modified FGF-1 comprises an N-Met residue and one or more mutations at positions 12, 16, 66, 117, and 134 of SEQ ID NO: 1. In one specific embodiment, an exemplary modified FGF-1 sequence, comprising an N-Met residue, is disclosed as SEQ ID NO: 2.

In some embodiments described herein, where the modified FGF-1 polypeptide is expressed with an N-terminal methionine (N-Met) residue, the polypeptide is subsequently purified without a step requiring proteolytic cleavage for removal of an N-terminal peptide. Accordingly, in some embodiments, the present disclosure provides a method of treating dry eye comprising administering a modified FGF-1 polypeptide that is prepared by a rapid purification method, without involving a proteolytic cleavage step for removal of an N-terminal peptide. In some instances, this is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from uncleaved material. In some embodiments, the modified FGF-1 polypeptides described herein, can have increased stability (e.g. thermostability), reduced number of buried free thiols, and/or increased effective heparan sulfate proteoglycan (HSPG) affinity.

Several other advantages are also associated with the use of the modified FGF-1 polypeptides in the methods described herein. For example, the modified FGF-1 polypeptides described herein can be administered without heparin in its pharmaceutical composition or formulation (e.g., an ophthalmic formulation), avoiding potential safety issues related to its biologic origin. In addition, avoidance of heparin allows the use of higher doses of the modified FGF-1 polypeptides without complications resulting from local heparin-induced adverse events or preexisting anti-heparin antibodies. Furthermore, in the absence of heparin, immediate binding of the modified FGF to tissue is maximized and systemic distribution is significantly reduced. The modified FGF-1 polypeptides described herein are also advantage of having enhanced local sequestration and reduced redistribution kinetics, thus increasing the elimination half-life and mean residence time (MRT) at the site of delivery, and allowing for a reduced dosing frequency. This can be the result of modified FGF-1 polypeptides described herein that have increased stability (e.g. thermostability), reduced number of buried free thiols, and/or increased effective heparan sulfate proteoglycan (HSPG) affinity.

The FGF-1 polypeptides of the present disclosure comprise, in various embodiments, modifications at the N-terminus of the polypeptide, such as an addition, a truncation, or a combination of additions and truncations. In some embodiments, the modification is the addition of a single N-terminal methionine residue. In some embodiments, the modification is the addition of an extension peptide. In some embodiments, the modification is a truncation of one or more of the first five residues of a FGF-1 polypeptide. In some embodiments, the FGF-1 polypeptides comprise a sequence as set forth in SEQ ID NO: 1, with one or more mutations, in addition to the N-terminal modification. Several examples of the modified FGF-1 polypeptides for use in the methods disclosed herein comprise an N-terminal methionine (N-Met) residue in a mature form of the polypeptide. The retention of biological activity when amino acids are added to the N-terminus of a protein is unpredictable. Some proteins are tolerant of this and some are not, and the retention of biological activity and the potential for changes in stability are only determined empirically. In some embodiments, the methods of this disclosure comprise administering a modified FGF-1 polypeptide comprising an N-terminal Met residue that retain biological activity and stability.

In some embodiments, the methods of this disclosure comprise administering a modified FGF-1 as described herein, comprising an N-Met residue in its mature form, that has at least similar biological activity as a version without the N-Met residue. N-terminal methionine removal, or excision, is a co-translational process that occurs as soon as a polypeptide emerges from the ribosome. The removal of the N-terminal methionine involves the substrate specificities of a cleavage enzyme, methionine aminopeptidase (metAP), which recognizes a methionine residue which is followed by an amino acid residue with a small side chain, such as alanine, glycine, proline, serine, threonine, or valine. Due to this substrate sequence specificity, the modified FGF-1 of the first embodiment, which comprises an N-Met residue followed by phenylalanine, see position 1 of SEQ ID NO: 1, is not processed by metAP. Thus, by expressing the modified FGF-1 with a methionine residue directly upstream of SEQ ID NO: 1, a mature modified FGF-1, comprising methionine as its N-terminal residue, can be obtained. In some embodiments, the modified FGF-1 according to the first embodiment is not expressed with an N-terminal peptide and therefore is not subject to proteolytic cleavage for removal of the same, during subsequent purification.

Provided herein, in some embodiments, is a method of treating dry eye comprising administering a modified FGF-1 polypeptide comprising the sequence set forth as SEQ ID NO: 1, with one or more mutations, wherein the modified polypeptide further comprises a methionine residue upstream to the first residue of SEQ ID NO: 1, and one or more amino acids of the peptide set forth as SEQ ID NO: 3. A peptide comprising one or more residues of SEQ ID NO: 3 is herein referred to as an “extension peptide.” Thus, the modified FGF-1 according to the second embodiment comprises the sequence set forth as SEQ ID NO: 1, with one or more mutations, a methionine residue upstream to the first residue of SEQ ID NO: 1, and an extension peptide positioned between the methionine residue and the first residue of SEQ ID NO:1. In some embodiments, the modified FGF-1 polypeptide comprising the N-terminal methionine and an extension peptide, positioned between the methionine residue and the first residue of SEQ ID NO: 1, is a mature form of the polypeptide. In some embodiments, the modified FGF-1 polypeptide comprises one or more mutations at positions 12, 16, 66, 117, and 134 of SEQ ID NO: 1, which polypeptide is expressed in a host cell with a methionine residue upstream to the first residue of SEQ ID NO: 1, and further an extension peptide positioned between the methionine residue and the first residue of SEQ ID NO: 1. In some embodiments, the modified FGF-1 polypeptide is expressed with an extension peptide comprising five residues of SEQ ID NO: 3, positioned between the methionine residue and the first residue of SEQ ID NO: 1. In some embodiments, the modified FGF-1 polypeptide is expressed with four residues of SEQ ID NO: 3, positioned between the methionine residue and the first residue of SEQ ID NO: 1. In some embodiments, the modified FGF-1 polypeptide is expressed with three residues of SEQ ID NO: 3, positioned between the methionine residue and the first residue of SEQ ID NO: 1. In some embodiments, the modified FGF-1 polypeptide is expressed with two residues of SEQ ID NO: 3, positioned between the methionine residue and the first residue of SEQ ID NO: 1. In some embodiments, the modified FGF-1 polypeptide is expressed with one residue of SEQ ID NO: 3, positioned between the methionine residue and the first residue of SEQ ID NO: 1. Exemplary sequences of the extension peptide include SEQ ID NOS: 4-8.

In some instances, the methods of this disclosure comprise administering a modified FGF-1 polypeptide comprising an extension peptide and an N-terminal methionine residue, is not subject to N-terminal processing for removal of the methionine residue, whereas in some instances the methionine is excised by a cleavage enzyme. Typically, the cleavage enzyme is methionine aminopeptidase (metAP). Thus, in some examples, the mature form of the modified FGF-1 polypeptide comprises an N-Met residue followed by an extension peptide as described herein. Exemplary sequences of mature forms of modified FGF-1 polypeptides comprising an N-terminal methionine, and one or more residues of the extension peptide, positioned between the methionine residue and the first residue of SEQ ID NO:1, are set forth as SEQ ID NOS: 9-13, wherein the sequences further comprise one or more mutations at amino acids corresponding to positions 12, 16, 66, 117, and 134 of SEQ ID NO: 1. Additional exemplar sequences of mature modified FGF-1 polypeptides comprising an N-terminal methionine, and an extension peptide are set forth as SEQ ID NOS: 14-18. In some other examples, the mature form of the modified FGF-1 polypeptide does not comprise an N-Met residue but includes only an extension peptide. Exemplary sequences of mature forms of modified FGF-1 polypeptides comprising an extension peptide, positioned upstream to the first residue of SEQ ID NO:1 are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide are set forth as SEQ ID NOS: 24-28. In some embodiments, the methionine residue is cleaved by metAP when the extension peptide starts with an alanine (as in SEQ ID NO: 4) or with a threonine (as in SEQ ID NO: 5). In those instances, the mature FGF-1 polypeptide does not comprise an N-terminal methionine residue, e.g., SEQ ID NOS: 19, 21, 24, and 26.

Provided herein, in some embodiments, is a method of this disclosure comprising administering a modified FGF-1 polypeptide comprising the sequence set forth as SEQ ID NO: 1, with one or more mutations, wherein the modified polypeptide further comprises an extension peptide positioned upstream to the first residue of SEQ ID NO: 1. In some embodiments, the modified FGF-1 polypeptide comprising an extension peptide is a mature form of the polypeptide. In some embodiments, the modified FGF-1 polypeptide comprising one or more mutations at positions 12, 16, 66, 117, and 134 of SEQ ID NO: 1, which polypeptide is expressed in a host cell with one or more amino acid residues of the extension peptide positioned upstream to the first residue of SEQ ID NO: 1. Exemplary sequences of the modified FGF-1 polypeptides comprising an extension peptide, expressed without an N-terminal methionine residue, are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide, and expressed without an N-terminal methionine residue, are set forth as SEQ ID NOS: 24-28.

In some embodiments, the modified FGF-1 polypeptide does not comprise an extension peptide between the N-terminal methionine residue and the first residue of SEQ ID NO: 1 or 206. In some embodiments, the modified FGF-1 polypeptide comprises an N-terminal methionine and a polypeptide of SEQ ID NO: 1 or 206, wherein the N-terminal methionine is directly in the positioned between the methionine residue and the first residue of SEQ ID NO: 1 or 206. In some embodiments, the modified FGF-1 polypeptide is a mature form of the polypeptide.

Provided herein, in some embodiments, is a method of this disclosure comprising administering a modified FGF-1 polypeptide comprising the sequence set forth as SEQ ID NO: 1, with one or more mutations, wherein the modified polypeptide further comprises a truncation of one or more of the first five residues of SEQ ID NO: 1. In some embodiments, the modified FGF-1 polypeptide comprising the truncation of one or more of the first five residues of SEQ ID NO: 1 is the mature form of the polypeptide. In some embodiments, the modified FGF-1 polypeptide comprises one or more mutations at positions 12, 16, 66, 117, and 134 of SEQ ID NO: 1, wherein one or more of the first five residues of SEQ ID NO: 1 is deleted. In some cases, the modified FGF-1 polypeptide comprising truncations is expressed with an N-terminal methionine residue. For instance, the modified FGF-1 polypeptide, in some embodiments, comprises a sequence wherein the N-Met residue is followed by the second residue, asparagine, of SEQ ID NO: 1. In some cases, the modified FGF-1 polypeptide comprises an N-Met residue followed by the third residue, leucine, of SEQ ID NO: 1. In some cases, the modified FGF-1 polypeptide comprises an N-Met residue followed by the fourth residue, proline, of SEQ ID NO: 1. In some cases, the modified FGF-1 polypeptide comprises an N-Met residue followed by the fifth residue, proline, of SEQ ID NO:1. An extension peptide can be positioned in between the N-Met residue and the first, second, third, fourth, or fifth residue of SEQ ID NO: 1. Examples of a mature form of the modified FGF-1 polypeptide wherein an N-Met residue is followed by the second, third, fourth, or fifth residue of SEQ ID NO: 1 are shown in SEQ ID NOS: 37-40, wherein the sequences further comprise one or more mutations at amino acids corresponding to positions 12, 16, 66, 117, and 134 of SEQ ID NO: 1. Additional examples of modified FGF-1 polypeptides comprising truncations and an N-Met residue, are provided in SEQ ID NOS: 41-44.

The present disclosure also relates to a method of administering a modified FGF-1 polypeptide comprising one or more mutations of SEQ ID NO: 1, wherein the polypeptide is expressed with an N-Met residue followed by an extension peptide, and the extension peptide is followed by truncation of one or more of the first five residues of SEQ ID NO: 1. In some embodiments, the modified FGF-1 polypeptide comprises one or more mutations at positions 12, 16, 66, 117, and 134 of SEQ ID NO: 1, wherein the polypeptide is expressed with an N-Met residue followed by an extension peptide, and the extension peptide is followed by truncation of one or more of the first five residues of SEQ ID NO: 1. Examples of such sequences expressed with an N-Met residue followed by an extension peptide, which extension peptide is followed by truncation of one or more of the first five residues of SEQ ID NO: 1 are disclosed as SEQ ID NOs: 45-68, wherein the sequences further comprise one or more mutations at amino acids corresponding to positions 12, 16, 66, 117, and 134 of SEQ ID NO: 1. In some examples, the N-terminal methionine is cleaved off by N-terminal processing and accordingly the mature form of the modified FGF-1 polypeptide comprises only one or more residues of the leader sequence followed by truncation of one or more of the first five residues of SEQ ID NO: 1, as exemplified in SEQ ID NOS: 69-92, wherein the exemplary sequences further comprise one or more mutations at amino acids corresponding to positions 12, 16, 66, 117, and 134 of SEQ ID NO: 1. Additional examples of sequences without N-Met residue but including an extension peptide and truncations of N-terminal residues, are provided in SEQ ID NO: 93, 94, and 96-117.

In some examples, the N-Met residue is retained in the mature modified FGF-1 polypeptide sequence, and accordingly the mature forms comprise sequences as exemplified in SEQ ID NO: 45-68, further comprising one or more mutations at amino acids corresponding to positions 12, 16, 66, 117, and 134 of SEQ ID NO: 1. Additional examples of sequences comprising an N-Met residue, an extension peptide and truncations of N-terminal residues, are provided in SEQ ID NO: 118-141 and 207.

The truncated versions of the modified FGF-1 polypeptides comprising one or more mutations at positions 12, 16, 66, 117, and 134 of SEQ ID NO: 1, are expressed without an N-terminal methionine residue, and further without an extension peptide. In some examples, mature modified FGF-1 polypeptides comprise a sequence as set forth in SEQ ID NOS: 29-32, wherein the sequences further comprise one or more mutations at amino acids corresponding to positions 12, 16, 66, 117, and 134 of SEQ ID NO: 1. In some examples, the modified FGF-1 polypeptides comprise a sequence selected from the group consisting of SEQ ID NOS: 33-36.

In instances where the modified FGF-1 polypeptide, or its truncated version, comprising one or more mutations at positions 12, 16, 66, 117, and 134 of SEQ ID NO: 1, is expressed with an N-terminal methionine followed by an extension peptide, the methionine residue is either retained or cleaved off of the N-terminus during maturation of the polypeptide after expression. In some examples, where the modified FGF-1 polypeptide is expressed with an alanine next to the N-Met residue, e.g., SEQ ID NO: 14, the methionine is cleaved, to yield a mature FGF-1 polypeptide that does not comprise an N-Met residue, e.g., SEQ ID NO: 19. In some examples, where the modified FGF-1 polypeptide is expressed with a threonine next to the N-Met residue, e.g., SEQ ID NO: 16, the methionine is cleaved, to yield a mature FGF-1 polypeptide that does not comprise an N-Met residue, e.g., SEQ ID NO: 20. In some examples, where the modified FGF-1 polypeptide is expressed with a glutamic acid next to the N-Met residue, e.g., SEQ ID NO: 17, the methionine is not cleaved, to yield a mature FGF-1 that comprise an N-terminal methionine and has the same sequence as the expressed form.

Provided herein, in some embodiments, is a method comprising administering a modified FGF-1 polypeptide comprising the sequence set forth as SEQ ID NO: 1, comprising a mutation at position 67. In some embodiments, the modified FGF-1 polypeptide comprises a mutation at position 67 of SEQ ID NO: 1, one or more further mutations at positions 12, 16, 66, 117, and 134, and is expressed with an N-Met residue. The internal methionine at position 67 can be replaced, for example, with an alanine residue. In absence of the internal methionine at position 67, the N-terminal methionine of the modified FGF-1 polypeptide can be cleaved, post-expression; using cyanogen bromide (CNBr), an agent that specifically cleaves the amide bond after methionine residues. In some cases, the modified FGF-1 polypeptides are expressed with an extension peptide. In some other cases, modified FGF-1 polypeptides are expressed in a form comprising truncations of one or more of the first five residues of SEQ ID NO: 1, as exemplified in SEQ ID NOS: 142-149, wherein the sequences further comprise one or more mutations at amino acids corresponding to positions 12, 16, 66, 117, and 134 of SEQ ID NO: 1. In yet other examples, the modified FGF-1 polypeptides are expressed in a form comprising an extension peptide and truncations of one or more of the first five residues of SEQ ID NO: 1, as exemplified in SEQ ID NOS: 151-175. Additional examples of the modified FGF-1 polypeptides, in their mature forms, are set forth in SEQ ID NOS: 174-204. Among the modified FGF-1 polypeptides expressed in a form that comprises an internal methionine mutation, in cases where the polypeptide is expressed with an N-terminal methionine followed by an alanine or a threonine residue from the extension peptide, e.g., SEQ ID NO: 175 and SEQ ID NO: 177, respectively, the N-terminal methionine can be cleaved off during maturation of the polypeptide either by metAP or using CNBr.

Provided herein, in some embodiments, is a method comprising administering a modified FGF-1 polypeptide comprising the sequence set forth as SEQ ID NO: 205, for use in a method as described herein. Provided herein, in some embodiments, is a method comprising administering a modified FGF-1 polypeptide comprising the sequence set forth as SEQ ID NO: 206, for use in a method as described herein.

The present disclosure further relates to methods comprising administering modified FGF-1 polypeptides comprising any combination of deletion, insertion, and substitution of SEQ ID NO: 1. Amino acid substitutions may be introduced into a modified FGF-1 polypeptide and the products screened for a desired activity, e.g., retained/improved effectivity in treating fibrotic diseases. Amino acid substitutions may also be introduced into a modified FGF-1 polypeptide and the products screened for a desired physicochemical property, e.g., less prone to aggregation, improved solubility, prolonged half-life, ease of formulating as an ophthalmic pharmaceutical, enhanced stability, improved shelf-life. Both conservative and non-conservative amino acid substitutions are contemplated.

In some instances, the modified FGF-1 polypeptide, as in any of the above embodiments, is expressed in a form that comprises at least 136 amino acids. In some embodiments, the modified FGF-1 polypeptide is expressed in a form that comprises 137 amino acids. In some embodiments, the modified FGF-1 polypeptide is expressed in a form that comprises 138 amino acids. In some embodiments, the modified FGF-1 polypeptide is expressed in a form that comprises 139 amino acids. In some embodiments, the modified FGF-1 polypeptide is expressed in a form that comprises 140 amino acids. In some embodiments, the modified FGF-1 polypeptide is expressed in a form that comprises 141 amino acids. In some embodiments, the modified FGF-1 polypeptide is expressed in a form that comprises 142 amino acids. In some embodiments, the modified FGF-1 polypeptide is expressed in a form that comprises 143 amino acids. In some embodiments, the modified FGF-1 polypeptide is expressed in a form that comprises 144 amino acids. In some embodiments, the modified FGF-1 polypeptide is expressed in a form that comprises 145 amino acids. In some embodiments, the modified FGF-1 polypeptide is expressed in a form that comprises 146 amino acids.

The modified FGF-1 polypeptide, as in any of the above embodiments, comprises at least 136 amino acids in the mature form. In some examples, the modified FGF-1 polypeptide comprises 137 amino acids in the mature form. In some examples, the modified FGF-1 polypeptide comprises 138 amino acids in the mature form. In some examples, the modified FGF-1 polypeptide comprises 139 amino acids in the mature form. In some examples, the modified FGF-1 polypeptide comprises 140 amino acids in the mature form. In some examples, the modified FGF-1 polypeptide comprises 141 amino acids in the mature form. In some examples, the modified FGF-1 polypeptide comprises 142 amino acids in the mature form. In some examples, the modified FGF-1 polypeptide comprises 143 amino acids in the mature form. In some examples, the modified FGF-1 polypeptide comprises 144 amino acids in the mature form. In some examples, the modified FGF-1 polypeptide comprises 145 amino acids in the mature form. In some examples, the modified FGF-1 polypeptide comprises 146 amino acids in the mature form.

In some embodiments, methods of this disclosure comprises administering a modified FGF-1 polypeptide comprises at least 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, provided that said polypeptide comprises an N-Met residue in the mature form of the polypeptide. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 9-13, provided that said polypeptide comprises the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 14-18, provided that said polypeptide comprises the N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 24-28, provided that said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 37-40, provided that said polypeptide comprises an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 41-44, provided that said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 45-68, provided that said polypeptide comprises one or more mutations at amino acid positions corresponding to positions 12, 16, 66, 117, and 134 of SEQ ID NO: 1, and said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 69-92, comprises one or more mutations at amino acid positions corresponding to positions 12, 16, 66, 117, and 134 of SEQ ID NO: 1, and said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 93, 94, and 96-117, provided that said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 118-141 and 207, provided that said polypeptide comprises an N-Met residue in its mature form. In some embodiments, the sequence of the modified FGF-1 polypeptide comprises 50%, 55%, 60%, 65%, 70%, 75% 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 29-32, provided that said polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55% 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 33-36. In some embodiments, the sequence of the modified FGF-1 polypeptide comprises 50%, 55%, 60%, 65%, 70%, 75% 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 142-204. In some embodiments, the modified FGF-1 polypeptide comprises 50%, 55%, 60%, 65%, 70%, 75% 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 12 with, for example, the mutation Lys12Val, and wherein said modified FGF-1 polypeptide comprises an N-terminal methionine in its mature form. In some embodiments, the modified FGF-1 polypeptide comprises a sequence with a mutation at position 12 of SEQ ID NO: 1, for example the mutation Lys12Val, and with truncation of one or more of the first five residue of SEQ ID NO: 1, wherein said modified FGF-1 polypeptide comprises an N-Met residue in its mature form. In some embodiments, the modified FGF-1 polypeptide comprises a sequence with a mutation at position 12 of SEQ ID NO: 1, for example the mutation Lys12Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 12 of SEQ ID NO: 1, for example the mutation Lys12Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 16 with, for example, the mutation Cys16Ser, and wherein said modified FGF-1 polypeptide comprises an N-met residue in its mature form. In some embodiments, the modified FGF-1 polypeptide comprises a sequence with a mutation at position 16 of SEQ ID NO: 1, for example the mutation Cys16Ser, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 16 of SEQ ID NO: 16, for example the mutation Cys16Ser, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at position 16 of SEQ ID NO: 1, for example the mutation Cys16Ser, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 66 with, for example, the mutation Ala66Cys, and wherein said modified FGF-1 polypeptide comprises an N-terminal methionine in its mature form. In some embodiments, the modified FGF-1 polypeptide comprises a sequence with a mutation at position 66 of SEQ ID NO: 1, for example the mutation Ala66Cys, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 66 of SEQ ID NO: 1, for example the mutation Ala66Cys, with an extension peptide, and with truncation of one or more of the first five residue of SEQ ID NO: 1, wherein said modified FGF-1 polypeptide is expressed with an N-Met residue. In some embodiments, the modified FGF-1 polypeptide comprises a sequence with mutations at position 66 of SEQ ID NO: 1, for example the mutation Ala66Cys, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 117 with, for example, the mutation Cys117Val, and wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys117Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 117 of SEQ ID NO: 1, for example the mutation Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 134 with, for example, the mutation Pro134Val, and wherein said modified FGF-1 polypeptide comprises an N-terminal methionine in its mature form. In some embodiments, the modified FGF-1 polypeptide comprises a sequence with a mutation at position 134 of SEQ ID NO: 1, for example the mutation Pro134Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 134 of SEQ ID NO: 1, for example the mutation Pro134Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 134 of SEQ ID NO: 1, for example the mutation Pro134Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at positions 16,66, and 117 of SEQ ID NO: 1, with, for example, the mutation Cys16Ser, Ala66Cys, and Cys117Val, and wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to a sequence selected from SEQ ID NOs: 2, 9-94, 96-204, or 207. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 205 or 206.

In some embodiments, the modified FGF-1 polypeptide is thermostable. As used herein, a thermostable FGF (e.g., a thermostable FGF-1) refers to an FGF having a modified amino acid sequence relative to SEQ ID NO: 1 that is also more stable than the polypeptide of SEQ ID NO: 1 under the same conditions. Examples of mutations capable of conferring thermostability to FGF (e.g., FGF-1) and methods for assessing thermostability are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 12 and/or 134 are mutated in FGF-1 to generate a modified FGF-1 that is thermostable. An FGF-1 formulation may be considered “stable” for a duration of time at a certain temperature, which is understood as the formulation in which the FGF-1 is present in its original purity and form for the designated period of time at the designated temperature. In some embodiments, the FGF-1 may be considered as remaining in its original purity and form, if there is less than 5%, less than 2% or less than 1% degradation or change in its monomeric form. Such a change may be detectable by any of the analytic procedures discussed herein, for example, chromatographic procedures, ELISA, SDS-PAGE and western blot.

In some embodiments, the modified FGF-1 polypeptide includes one or more modifications that reduce the number of reactive thiols (e.g., free cysteines). Examples such modifications in FGF-1 are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 83 and/or 117 are mutated in SEQ ID NO: 1 to generate a modified FGF-1 that reduces the number of reactive thiols. In some embodiments, the modified FGF includes one or more modifications that enable formation of an internal disulfide linkage. In some embodiments, position 66 is mutated in SEQ ID NO: 1 to generate a modified FGF-1 that comprises an internal disulfide linkage.

In some embodiments, the modified FGF-1 polypeptides described herein can be administered without exogenous heparin in the formulation for stability, they can be formulated and applied without heparin and thus are more able to bind to the tissue heparans. Such modified FGF-1 polypeptides have a high affinity for tissue heparans that are exposed in a surgical, traumatic or dystrophic conditions and disease-states and so bind to diseased tissue on application. In addition, the modified FGF-1 polypeptides being more thermally stable are suitable for formulation and storage at room temperature. The stability of the modified FGF-1 polypeptides also makes them suitable for administration in both solution (e.g., immediate release) and sustained-release formulations.

In some embodiments, the modified FGF-1 polypeptide is SEQ ID NO: 1 that has been modified at one or more of positions 12, 16, 66, 117, and 134. In some embodiments, the modified FGF is SEQ ID NO: 1 that has been modified at positions 16, 66, and 117. The amino acid positions can be substituted with, e.g., Ser, Cys, Val, or other amino acids to create disulfide linkages between modified amino acids and wild-type amino acids. In some embodiments, the modified FGF comprises the amino acid sequence of SEQ ID NO: 2, also referred to as N-Met THX1114. In some embodiments, the modified FGF-1 polypeptide comprises one or more mutations selected from the group consisting of: Lys12Val, Pro134Val, Ala66Cys, Cys117Val, and Pro134Val. In some embodiments, the modified FGF-1 polypeptide comprises the sequence of SEQ ID NO: 2.

In some embodiments, the modified FGF-1 polypeptides or compositions described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. The modified FGF-1 polypeptides described herein may be labeled isotopically (e.g., with a radioisotope) or by other means, including, but not limited to, the use of chromophores or fluorescent moieties, bioluminescent labels, photoactivatable or chemiluminescent labels. The present discloser further relates to modified FGF polypeptides comprising N-terminal modification(s), wherein the modified FGF polypeptide can be any member of the FGF family, including FGF-1 (SEQ ID NO: 1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23, and FGF-24. In some embodiments, the synthesis of modified FGF-1 polypeptides as described herein is accomplished using means described in the art, using the methods described herein, or by a combination thereof. In some embodiments, the sequence of the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at one or more positions 16, 66, and 117 with, for example, the mutations Cys16Ser, Ala66Cys, and Cys117Val. In some embodiments, the modified FGF comprises the wild-type human FGF-1 sequence with a mutation at positions 16, 66 and 117, for example the mutations Cys16Ser, Ala66Cys, and Cys117Val.

### Recombinant Techniques for Preparation of Modified FGF-1 Polypeptides

A variety of host-expression vector systems may be utilized to produce the modified FGF-1 polypeptides provided herein for use in methods of this disclosure. Such host-expression systems represent vehicles by which the modified FGF-1 polypeptides may be produced and subsequently purified, but also represent cells that may, when transformed or transfected with the appropriate nucleotide coding sequences, exhibit the modified gene product in situ. Examples of host-expression systems include but are not limited to, bacteria, insect, plant, mammalian, including human host systems, such as, but not limited to, insect cell systems infected with recombinant virus expression vectors (e.g., baculovirus) containing nucleotide sequences coding for the modified FGF-1 polypeptides; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences for the modified FGF-1 polypeptides; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter, or from yeast-derived plasmids e.g. pSH19 and pSH15, or from bacteriophages such as lambda phase and derivatives thereof. Examples of bacterial expression systems include but are not limited to Escherichia coli-derived plasmids (e.g., pBR322, pBR325, pUC12, pUC13, and pET-3); Bacillus subtilis-derived plasmids (e.g., PUB110, pTP5, and pC194). In some embodiments the bacterial expression system comprises a pMKet vector. In some embodiments, a method comprising use of the pMKet bacterial expression vector to express modified FGF-1 polypeptide improves yield of the modified FGF-1 by about 5 fold to about 60-fold, compared to a method comprising subcloning a sequence encoding the modified FGF-1 into a pET vector. In some embodiments, the method comprising the subcloning of modified FGF-1 in a pET vector results in an yield of about 0.5 g-about 0.7 g/100 L following a fermentation run. In some embodiments, the method comprising the subcloning of modified FGF-1 in a pMKet vector results in an yield of about 20 g-about 40 g/100 L following a fermentation run, for example 37 g/100 L. In some embodiments, the method comprising the subcloning of modified FGF1 in a pMKet and purifying the protein from the vector using the techniques described herein results in a yield of about 82 g/50 L.

In some embodiments, a host cell strain is chosen such that it modulates the expression of the inserted sequences, or modifies and processes the gene product in the specific fashion desired. Such modifications and processing of protein products may be important for the function of the protein. Different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and WI38.

For long-term, high-yield production of recombinant peptides, stable expression is desired. For example, cell lines that stably express the recombinant modified FGF-1 polypeptides may be engineered. In some embodiments, rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. In some examples, this method may advantageously be used to engineer cell lines that express the modified FGF-1 polypeptide product. Such engineered cell lines may be particularly useful in screening and evaluation of compounds that affect the biological of the gene product.

In some embodiments, bacterial cells are used for expressing the recombinant FGF protein. In some embodiments the bacterial cell is an Escherichia coli cell (E. coli). In some embodiments, the E. coli strain is selected from BLA21A1, K12 HMS174, and W3110. For long-term, high-yield production of recombinant peptides, stable expression is desired. For example, cell lines that stably express the recombinant modified FGF-1 polypeptides may be engineered. In some embodiments, rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. In some embodiments, the recombinant nucleic acid comprising a sequence encoding the FGF-1 polypeptide optimized for maximizing codon usage by the strain or cell in which it is expressed. In some embodiments, the recombinant nucleic acid comprising a sequence encoding the FGF-1 polypeptide is operably linked to a promoter, 3′ UTR regulatory sequence, for example a poly A sequence or a sequence that stabilizes the transcript, and helps in translation. In some embodiments the plasmid vector comprises a selection marker, such as an antibiotic resistance gene, such as kanamycin.

In some embodiments the promoter for bacterial expression is a T7 promoter. In some embodiments the promoter for bacterial expression is a Tac promoter. In some embodiments, the plasmid comprises a pBR322 on sequence. In some embodiments, the bacterial expression vector is modified from a commercially available vector backbone, such as pBR322, pBR325, pUC12, pUC13, and pET-T3, or pET-T7 vectors.

In some embodiments, a periplasmic expression of the protein is intended. The recombinant protein may accumulate in inclusion bodies. In some embodiments, the cytoplasmic expression of the protein is intended. In some embodiments, the cytoplasmic expression of the protein is intended, wherein the protein is an insoluble protein. In some embodiments, the recombinant polypeptide may be desired for extracellular release. In some embodiments, the recombinant nucleic acid encoding the polypeptide may comprise a suitable leader sequence, such as an ompA leader sequence. In some embodiments, the recombinant nucleic acid encoding the modified FGF-1 polypeptide does not comprise a leader sequence, such as an ompA leader sequence. In some embodiments, during manufacture, the modified FGF-1 polypeptide, in certain step, is directed to the periplasmic space. The periplasmic space comprises inclusion bodies, where the polypeptide is likely to accumulate. Inclusion bodies may then be harvested after cell fractionation to recover the polypeptide. In some embodiments, the recombinant nucleic acid does not contain a leader sequence. In some embodiments the modified FGF-1 is directed for cytoplasmic expression in the cell.

In some embodiments, the recombinant nucleic acid construct comprises one or more modification for increasing yield of the modified FGF-1 polypeptide from the cell. In some embodiments, the one or more modifications comprise sequence optimization for increased expression of the modified FGF-1 polypeptide in the cell. In one embodiment, the one or more modifications comprise modifications in the plasmid. In one embodiment, the one or more modifications comprise selecting a suitable promoter for increasing yield of the modified FGF-1 polypeptide from the cell.

In further embodiments, one or more modifications are considered towards developing the host cell for expressing the polypeptide. In some embodiments, one or more modifications may be considered resulting in adjusting the adequate nutrient media for maximizing cell proliferation. In one embodiment, the adequate nutrient media comprises a carbon source. In one embodiment, the carbon source is glucose or glycerol.

In one embodiment, one or more modifications are made in the plasmid to increase the copy number and expression efficiency of the plasmid in the host cell. In one embodiment, one or more modifications are considered for maximizing the yield of the modified FGF-1 polypeptide from the cell, wherein the one or more modifications may be selected from:
i. modification within the recombinant nucleic acid encoding the mutant FGF-1 polypeptide;
ii. modification within the recombinant nucleic acid comprising one or more regulatory elements related to the recombinant nucleic acid encoding the mutant FGF-1 polypeptide, selected from a promoter, an enhancer, a 5′-untranslated region, a 3′-untranslated region, a poly A tail, a transcript stabilizing element;
iii. modification of the plasmid comprising the recombinant nucleic acid;
iv. modification of the cell strain or selection of a cell strain for maximizing cell proliferation;
v. modification of the cell growth media; and
vi. modifications in the processes of recovering the modified FGF-1 polypeptide from the cell.

In some embodiments, the bacterial cells are electroporated or chemically transformed with a plasmid comprising a recombinant nucleic acid comprising a sequence encoding the FGF-1 polypeptide. Following the introduction of the foreign DNA, engineered cells may be allowed to grow for 1-2 days in an enriched media, and then are switched to a selective media. In some embodiments, one or more carbon sources are used for maximizing the bacterial cell growth within a period of time for expansion of the expressed FGF polypeptide for increased production. In some embodiments the carbon source for the bacterial cell may be glucose. In some embodiments the carbon source for the bacterial cell may be glycerol.

The selectable marker in the recombinant plasmid confers resistance to the selection and allows cells to stably integrate the plasmid into their chromosomes and grow to form foci that in turn can be cloned and expanded into cell lines. In some examples, this method may advantageously be used to engineer cell lines that express the modified FGF-1 polypeptide product. Such engineered cell lines may be particularly useful in screening and evaluation of compounds that affect the biological of the gene product.

The method of production described herein can be easily scaled up to form large scale productions of bacterial cultures expressing the modified FGF-1. In some embodiments, the method can be scaled to produce FGF-1 in 1 L bacterial cultures, or in 10 L bacterial cultures, or in 100 L bacterial cultures, or in 500 L bacterial cultures. In some embodiments, the method is scalable to produce 1 g of modified FGF-1 polypeptide per batch. In some embodiments, the method is scalable to produce 10 g of modified FGF-1 polypeptide per batch. In some embodiments, the method is scalable to produce 100 g of modified FGF-1 polypeptide per batch. In some embodiments, the method is scalable to produce 1 Kg of modified FGF-1 polypeptide per batch. In some embodiments, the method is scalable to produce 10 Kg of modified FGF-1 polypeptide per batch. In some embodiments, the method is scalable to produce 100 Kg of modified FGF-1 polypeptide per batch.

In general, a seed culture is formed from transformed bacterial cells. The inoculated seed flasks may be incubated at about 235 RPM and 37° C. Following a 10-14 h incubation, a sample of the seed culture from each of the flasks may be tested for purity (microscopic observation of a wet mount with no contaminating organisms observed), pH, optical density at 600 nm (OD₆₀₀), and sterility hold. The seed cultures are desired to exhibit optimal growth by demonstrating an OD₆₀₀≥1.0 and no contaminating organisms. Six of the seed flasks from each production run may be selected for scale-up. Selection criteria may include a growth time of 12±2 h, an OD₆₀₀≥1.0, and having six flasks. To create the fermentor inoculum, the contents of the six flasks may be pooled into a 10 L bag (sterile single-use bioprocess container) in a BSC for a total seed culture volume of approximately six liters.

One or more 150 L fermentors may be prepared for the fermentation of cultured E. coli expressing mFGF-1 with production medium (comprising nutrients, for example, soytone 12 g/L, yeast extract 24 g/L, glycerol 15.1 g/L, potassium phosphate dibasic 12.5 g/L, potassium phosphate monobasic 3.8 g/L, and P2000 antifoam 0.1 mL/L). The production medium is sterilized in situ. The sterile medium is then supplemented with a 5±0.1 L sterile solution that contained magnesium sulfate heptahydrate 0.4 g/L, kanamycin 0.050 g/L. The one or more than one fermentors may be inoculated with six liters of pooled seed culture at the appropriate time. Fermentation cultures are then monitored every 60±30 min and the samples were processed for pH, purity (microscopic observation of a wet mount), and OD₆₀₀. Dissolved oxygen may be maintained by controlling agitation and air flow rates. The pH may be maintained within the desired range by making appropriate aseptic additions of phosphoric acid and/or ammonium hydroxide.

In some embodiments, the expression of mFGF-1 induced with addition of 0.2-0.4 g/L isopropyl-β-D-1-thio-galactopyranoside (IPTG) and 5.0 g/L L-Arabinose when the culture reaches an OD₆₀₀ of ˜4.5 at 3 hours post-inoculation. In some embodiments, a concentration of about 1 mM or 0.25 mM IPTG is used for induction, with or without kanamycin. Induction in the presence of kanamycin, in some embodiments, is not required to increase plasmid retention and cell viability, but, affects the production of the modified FGF-1 polypeptide. The length of induction, in some instances, is from about 8-12 hours, about 10-20 hours, about 20 hours, or about 24 hours. Yield of the modified FGF-1 polypeptide (as measured by final OD/cell paste (g/L)) in the presence of kanamycin, in some instances at a 1 L scale is about 1.1 to about 5 times, e.g., 1.2 times, greater than in the absence of kanamycin. For induction, in some embodiments, the carbon source is glycerol, e.g., at a concentration of at least about 30 g/L, temperature is about 37° C., and pH about 6.8. After induction, the fermentations may be continued for an additional three hours. Prior to centrifugation samples may be taken from each fermentor for analysis by SDS-PAGE and culture purity. The fermentations may be evaluated intermittently and may be grown for another 1-20 hours. In some embodiments, the final culture reaches an optical density of 50-100, 50-200, 50-250, 70-260 or about 100, 200 or 250.

In some embodiments, the harvesting of the lots may be performed by transferring the fermentation broth to the centrifuge via a peristaltic pump and tubing (at 0.5-0.8 liters per minute) and centrifuged at 20,000×g while cooling using a water-circulation jacket. The mass of the harvested cell paste may be measured, collected, divided into four containers, and placed in a ≤−70° C. freezer.

Cell Lysis: Frozen cells comprising the modified FGF-1 may be thawed at a suitable time, and resuspended in a suitable buffer, for example, the buffer may comprise Tris and EDTA. In an exemplary embodiment, the cells may be thawed in TES buffer (50 mM Tris, 20 mM EDTA, 100 mM NaCl, pH7.4) containing 1 mM DTE at a ratio of 1:5 (w/v), i.e. 1 gram of cell paste in 5 mL of buffer. The suspension may be chilled to below 16° C. before running through a high-pressure homogenizer. OD₆₀₀ is monitored after each pass until no significant decrease. An equal volume of TES+5% Triton X-100 may then be mixed into the ruptured-cell suspension. The ruptured cell suspension may be used for recovery of the expressed FGF-1 proteins. The expressed protein may be collected from the ruptured cell by passing the lysate through a specific capture method, such as affinity column packed with an agarose based resin (e.g., a highly cross-linked agarose based resin, such as, Capto™ DeVirs (Cytiva, BPG 300×500, Part #17-5466)), that specifically binds the FGF-1 protein, and is later eluted. However, for maximizing the FGF-1 recovery and yield, the protein may be directed for expression in the IBs, and the protein may be collected from the inclusion bodies. In an exemplary method, the mixture may be centrifuged at 15,900×g for 60 min at 4° C. to collect the mFGF-1-containing inclusion bodies. Following lysis, the overexpressed proteins may be recovered from inclusion bodies (IB) from E. coli paste by centrifugation.

In some embodiments, the capture method using an affinity column packed with an agarose based resin (e.g., Capto™ DeVirs) increases percent yield of the collected FGF-1 polypeptide by about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, in comparison to other capture methods.

Recovery from inclusion bodies: In an exemplary embodiment, for the recovery of FGF-1, the cell paste may be thawed at 2-8° C., resuspended in a suitable buffer. The buffer may comprise Tris and EDTA. For example, the cell paste may be thawed in 4.5 L of TES buffer, pH 7.4 (50 mM Tris, 100 mM NaCl, 20 mM EDTA) and the cells may then be lysed by pressure homogenization. Five homogenization passes (approximately 8,000 psi) may be performed to achieve maximum cell lysis. An equal volume of TES buffer and 5% Triton X-100, pH 7.4 may then be added to the lysate to obtain a 2.5% Triton concentration.

The mixture may be divided into 6-20 centrifuge bottles, as per convenience, which are then incubated for at least 30 min at approximately 15-20° C. with shaking at 225 RPM. The bottles may be centrifuged for 60 min at 15,900×g and 4° C. The supernatant was discarded as waste. Using a tissue homogenizer (Model Omni GLH850), the recovered inclusion bodies were individually resuspended in TE Buffer (˜1 L, 50 mM Tris, 20 mM EDTA, pH 7.4) with 2.5% (w/v) Triton X-100, and the bottles were incubated for at least 30 min at 15-20° C. with shaking at 225 RPM. After incubation, the bottles were centrifuged for 45 min at 15,900×g and 4° C. The inclusion body washing process suspension, incubation, and centrifugation was performed a total of three times. The recovered inclusion bodies may be stored overnight at 2° C.-8° C. The inclusion bodies were washed with TE buffer without Triton (˜1 L, 50 mM Tris, 20 nM EDTA, pH 7.4) and the bottles were incubated for at least 15 min at 15-20° C. with shaking at 225 RPM. In some embodiments, the IBs may be washed in a buffer comprising polysorbate 20 or polysorbate 80. After incubation, the bottles were centrifuged for 30 min at 15,900×g and 4° C. Washing without Triton, incubation, and centrifugation may be performed a total of five times. Samples may be removed and submitted for total protein and SDS-PAGE Coomassie Stain/densitometry analysis at this stage. In some embodiments, a total of 100, 200, 300, 400, 500, 600, 700, 800, 900 or 1,000 g of inclusion bodies may be recovered. The centrifuge bottles containing the washed inclusion bodies for each lot may be stored at ≤−70° C.

Solubilization of Washed Inclusion Bodies: Solubilization of the washed inclusion bodies for each lot may be performed using a solubilization buffer. A solubilization buffer may comprise chaotropic components, such as urea, or guanidine salts. The washed inclusion bodies may be removed from storage and thawed at 2-8° C. (15 h-19 h). Inclusion bodies may be centrifuged at 15,900×G, 4° C. for 60 min, and after removing the liquid phase the net weight of the pellets was determined. The inclusion bodies may then be solubilized in buffer. An exemplary solubilization buffer may comprise 4-8M Guanidine. An exemplary solubilization buffer may comprise 6M Guanidine. An exemplary solubilization buffer may comprise 4-6M Urea. Additionally, such a buffer may comprise 100 mM Tris, 2 mM EDTA, (pH 8.0). The buffer may be mixed at 10 mL per g ratio at 2-8° C. using a tissue homogenizer (e.g., Model Omni GLH850) at 10,000 RPM until the solution was visually homogenous. Guanidine is a chaotropic agent that results on protein denaturation. Dithioerythritol (DTE) may be used to a final concentration of 10 mg/mL to the solubilized inclusion bodies at 2-8° C. to reduce disulfide bonds to thiols. This reaction may continue for 2-6 hours. The solubilized inclusion bodies may then be centrifuged for 40 min at 15,900×g and 4° C. The complete process including centrifugation may be less than five hours. The supernatant may be collected into 2 L PETG bottles and stored at 2-8° C. (25-50 min) while the protein concentration was tested. Based on the protein results, the solubilized inclusion bodies may be diluted to a target concentration of 2.0±0.5 mg/mL with dilution buffer (6M Guanidine, 100 mM Tris, 2 mM EDTA, pH 8.0). DTE may be added to a target concentration of 10 mg/mL and mixed. The solubilized inclusion bodies may be stored at ≤−70° C. The IBs may be solubilized in 6 M guanidine hydrochloride in 100 mM Tris, 2 mM EDTA, pH 8.0 at a ratio of 10 mL buffer per gram IB. DTE may be added (10 mg/ml) and after 3-5 h mixing (initially with tissue homogenizer, Polytron PT 3100, followed by magnetic stir bar), the mixture may be centrifuged (15,900×g≥40 min). The supernatant may be filtered through a 0.45 μm filter.

Refolding of denatured protein: Guanidine-solubilized IBs (2±0.5 mg/mL) may be added into cold refolding buffer. The refolding buffer may comprise L-arginine. (e.g., 0.5 M L-Arginine, 100 mM Tris, 2 mM EDTA, pH 9.5). In some embodiments the refolding buffer may contain oxidized glutathione. In some embodiments, the refolding buffer may contain reduced glutathione. The solubilized mFGF-1 may be added slowly, e.g., dropwise into the vortex of the refold solution and mixing continued at 2-8° C. for 2 h. An equal volume of 3M ammonium sulfate may be added to the refolding solution and stirred at 2-8° C. for 1 h.

The recovered protein may be detected by SDS-PAGE. Total protein content may be measured by one or more methods known in the art. For example, total protein content may be measured by Coomassie stain of proteins resolved in SDS-PAGE. The recovered protein may be further purified using HPLC, for example size exclusion chromatography (SEC)-HPLC.

The biological activity of the protein may be assessed by cell proliferation assay in vitro. For this purpose, endothelial cell lines may be used. In some embodiments, fibroblasts may be used for in vitro proliferation assay.

In some embodiments one or more modifications are made to improve the recovery yield of the modified FGF-1 from the cell. The one or more improvements comprise improvements of the plasmid vector; improvement in choice of a suitable bacterial strain; improvement in the growth media; improvement in induction time with IPTG; improvement in incubation time for maximal growth of the bacteria; and optimizing the temperature for growth of the bacteria. In some embodiments, the one or more modifications lead to at least 2 fold, at least 3 fold, at least 4 fold, to at least 6 fold, at least 7 fold, at least 8 fold, to at least 9 fold, at least 10 fold, at least 12 fold, at least 15 fold, to at least 20 fold, at least 25 fold, at least 30 fold or at least 50 fold increase in the yield of modified FGF-1.

### Disulfide Bond Formation in Modified FGF-1 Polypeptides

In some embodiments, the modified FGF-1 polypeptide of the present disclosure comprises the following mutations in SEQ ID NO: 1—Cys16Ser, Ala66Cys, and Cys117Val, wherein the polypeptide includes an internal disulfide bond between the cysteine residues at positions 66 and 83. For many recombinant proteins, the formation of correct disulfide bonds is vital for attaining their biologically active three-dimensional conformation. The formation of erroneous disulfide bonds can lead to protein misfolding and aggregation into inclusion bodies. In E. coli, cysteine oxidation typically takes places in the periplasm, where disulfide bonds are formed in disulfide exchange reactions catalyzed by a myriad of enzymes, mainly from the Dsb family (Rosano, G. L., & Ceccarelli, E. A. (2014). Recombinant protein expression in Escherichia coli: advances and challenges. Frontiers in Microbiology, 5, 172). By contrast, disulfide bond formation in the cytoplasm is rare. This situation affects the production of recombinant proteins with disulfide bonds that are produced in the cytoplasm, such as a modified FGF-1 polypeptide comprising an internal disulfide linkage between Cys66 and Cys83. Accordingly, in some examples, an engineered E. coli strain that possess an oxidative cytoplasmic environment that favors disulfide bond formation is selected as a host cell for expression of the modified FGF-1 polypeptides (Rosano, G. L., & Ceccarelli, E. A. (2014). Recombinant protein expression in Escherichia coli: advances and challenges. Frontiers in Microbiology, 5, 172). Examples of such strains include but are not limited to Origami (Novagen), which has a trxB-gor-genotype in the K-12 background, and SHuffle® T7 Express strain (NEB), which has a trxB-gor-genotype in a BL21(DE3) background and constitutively expresses a chromosomal copy of the disulfide bond isomerase DsbC. It has been shown that DsbC promotes the correction of mis-oxidized proteins into their correct form and is also a chaperone that can assist in the folding of proteins that do not require disulfide bonds. Without being bound by a particular theory, it is contemplated that due to the action of DsbC, less target protein, such as the modified FGF-1 polypeptide comprising an internal disulfide linkage between Cys66 and Cys83, aggregates into inclusion bodies. Thus, in certain embodiments, the present disclosure identifies an improved method for cytoplasmic production of a modified FGF-1 polypeptide comprising internal disulfide linkage between Cys16 and Cys83.

In some embodiments where the modified FGF-1 polypeptide is expressed with an N-Met residue, the polypeptide is subsequently purified without a step requiring proteolytic cleavage for removal of an N-terminal peptide. Accordingly, in some embodiments, the present disclosure provides a method of rapid purification of the modified FGF-1 polypeptides described herein, without involving a proteolytic cleavage step for removal of an N-terminal peptide. This is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from uncleaved material.

### Methods of Use

Provided herein, in one embodiment, is a method of treating a disorder or condition in a subject comprising administering to the subject a modified FGF-1 polypeptide as described in the above embodiments, wherein the disease, disorder, or condition is dry eye, e.g., associated with at least one of: meibomian gland dysfunction, lacrimal gland insufficiency, lacrimal duct obstruction, reflex hyposecretion, Sjogren's syndrome, eyelid aperture disorder, blink disorder, or ocular surface disorders.

Provided herein are methods of treating dry eye by administering to a subject in need thereof a pharmaceutical composition comprising a therapeutically effective amount of a modified FGF-1 polypeptide. In some embodiments, the dry eye is caused by at least one of meibomian gland dysfunction (MGD), lacrimal gland insufficiency, lacrimal duct obstruction, reflex hyposecretion, Sjogren's syndrome, eyelid aperture disorder, blink disorder, or ocular surface disorders. When dry eye occurs with autoimmune diseases other than Sjogren's syndrome, it is sometimes called secondary Sjogren's syndrome. Dry eye in Sjogren's syndrome and other autoimmune diseases is caused by inflammation of the lacrimal gland. In some embodiments, the disclosure provides methods of treating dry eye caused by primary Sjogren's syndrome or secondary Sjogren's syndrome by administering to a subject in need thereof a pharmaceutical composition comprising a therapeutically effective amount of a modified FGF-1 polypeptide.

In some embodiments, the method comprises treating meibomian gland dysfunction (MGD), by administering a pharmaceutical composition comprising a therapeutically effective amount of a modified FGF-1 polypeptide described herein. In some embodiments, the method comprises treating Graft Versus Host Disease (GVHD) associated dry eye, by administering a pharmaceutical composition comprising a therapeutically effective amount of a modified FGF-1 polypeptide described herein. In some embodiments, the method comprises treating radiation-induced dry eye, by administering a pharmaceutical composition comprising a therapeutically effective amount of a modified FGF-1 polypeptide described herein. In some embodiments, the method comprises treating an age-related dry eye disease, by administering a pharmaceutical composition comprising a therapeutically effective amount of a modified FGF-1 polypeptide described herein. In some embodiments, the method comprises treating dry eye syndrome (DES) caused by lacrimal insufficiency, by administering a pharmaceutical composition comprising a therapeutically effective amount of a modified FGF-1 polypeptide described herein. In some embodiments, the method comprises treating keratoconjunctivitis sicca (KCS), by administering a pharmaceutical composition comprising a therapeutically effective amount of a modified FGF-1 polypeptide described herein. In some embodiments, the method comprises treating dry eye caused by Sjogren's syndrome (SS), by administering a pharmaceutical composition comprising a therapeutically effective amount of a modified FGF-1 polypeptide described herein. In some embodiments, the method comprises treating dry eye caused by secondary Sjogren's syndromes, by administering a pharmaceutical composition comprising a therapeutically effective amount of a modified FGF-1 polypeptide described herein. In some embodiments, the method comprises treating dry eye caused by Stevens-Johnson syndrome, by administering a pharmaceutical composition comprising a therapeutically effective amount of a modified FGF-1 polypeptide described herein. In some embodiments, the method comprises treating dry eyes caused by ocular cicatricial pemphigoid, by administering a pharmaceutical composition comprising a therapeutically effective amount of a modified FGF-1 polypeptide described herein. In some embodiments, the method comprises treating dry eye caused by corneal injury, by administering a pharmaceutical composition comprising a therapeutically effective amount of a modified FGF-1 polypeptide described herein. In some embodiments, the method comprises treating dry eye caused by ocular surface infection, by administering a pharmaceutical composition comprising a therapeutically effective amount of a modified FGF-1 polypeptide described herein. In some embodiments, the method comprises treating dry eyes caused by Riley-Day syndrome, by administering modified FGF-1 polypeptides described herein. In some embodiments, the method comprises treating dry eyes caused by congenital alacrimia, by administering a pharmaceutical composition comprising a therapeutically effective amount of a modified FGF-1 polypeptide described herein. In some embodiments, the method comprises treating dry eyes caused by nutritional disorders or deficiencies (including vitamin deficiencies), by administering a pharmaceutical composition comprising a therapeutically effective amount of a modified FGF-1 polypeptide described herein. In some embodiments, the method comprises treating dry eyes caused by pharmacologic side effects, by administering a pharmaceutical composition comprising a therapeutically effective amount of a modified FGF-1 polypeptide described herein. In some embodiments, the method comprises treating dry eyes caused by glandular destruction or tissue destruction, by administering a pharmaceutical composition comprising a therapeutically effective amount of a modified FGF-1 polypeptide described herein. In some embodiments, the method comprises treating dry eye caused by autoimmune and other immunodeficient disorders or inability to blink in comatose patients, by administering a pharmaceutical composition comprising a therapeutically effective amount of a modified FGF-1 polypeptide described herein. In some embodiments, the method of treating dry eye caused by environmental exposure to airborne particulates, smoke, smog, and excessively dry air, contact lens intolerance, by administering a pharmaceutical composition comprising a therapeutically effective amount of a modified FGF-1 polypeptide described herein. In some embodiments, the method comprises treating dry eyes caused by as a side effect of laser assisted vision correction procedures such as photorefractive keratectomy (PRK), laser-assisted sub-epithelial keratectomy (LASEK) and laser-assisted in situ keratomileusis (LASIK), by administering a pharmaceutical composition comprising a therapeutically effective amount of a modified FGF-1 polypeptide described herein. In some embodiments, the method comprises treating dry eye caused by virus infection or bacterial infection, by administering a pharmaceutical composition comprising a therapeutically effective amount of a modified FGF-1 polypeptide described herein. In some embodiments, the method comprises treating dry eye caused by infection by virus comprising HIV (trigger for AIDS), Mumps virus, Epstein-Barr virus (trigger for Pfeiffer's disease), Influenza (trigger for real flu), or Measles virus, by administering a pharmaceutical composition comprising a therapeutically effective amount of a modified FGF-1 polypeptide described herein. In some embodiments, the method comprises treating dry eye caused by skin diseases, by administering a pharmaceutical composition comprising a therapeutically effective amount of a modified FGF-1 polypeptide described herein.

In some embodiments, the disease, disorder or condition to be treated is dry eye caused by at least one of: meibomian gland dysfunction, lacrimal gland insufficiency, lacrimal duct obstruction, reflex hyposecretion, Sjogren's syndrome, eyelid aperture disorder, blink disorder, or ocular surface disorders. Examples of disease that causes and/or is associated with dry eye include but are not limited to: meibomian gland dysfunction (MGD) or Sjogren's syndrome (SS).

In some embodiments, dry eye is caused by meibomian gland dysfunction (MGD). Meibomian glands are the tiny oil glands which line the margin of the eyelids (the edges which touch when the eyelids are closed). These glands secrete oil which coats the surface of our eyes and keeps the water component of our tears from evaporating. In the early stages of Meibomian gland dysfunction (MGD), patients are often asymptomatic, but if left untreated, MGD can cause or exacerbate dry eye symptoms and eyelid inflammation. The oil glands become blocked with thickened secretions. Chronically clogged glands eventually become unable to secrete oil which results in permanent changes in the tear film and dry eyes. In some embodiments, a pharmaceutical composition comprising a therapeutically effective amount of a modified FGF-1 polypeptide of this disclosure is used to induce secreting enough oil from meibomian gland.

In some embodiments, dry eye is caused by Sjogren's syndrome. Sjogren's syndrome is a rheumatic autoimmune disease in which the endogenous defense system attacks exocrine glands (salivary and lacrimal glands) resulting in clinical symptoms of dry mouth and dry eye. In pathogenesis of SS, activated T-cells and B-cells infiltrate the lacrimal glands and autoimmune process leading to cell destruction. This process causes hyposecretion of tears and aqueous-deficient dry eye disease. The Sjogren's syndrome is the second most common autoimmune rheumatic disease, affecting between three and six per 100,000 Americans. The peak incidence is between the ages of 40 and 60, with a higher predilection in women than men (9 to 1). In some embodiments, a pharmaceutical composition comprising a therapeutically effective amount of a modified FGF-1 polypeptide of this disclosure is used to treat dry eye caused by Sjogren's syndrome by reducing macrophage infiltration in ocular tissue.

Sjogren's syndrome occurs in a primary form not associated with other diseases and in a secondary form associated with other autoimmune rheumatic conditions, including rheumatoid arthritis, systemic lupus erythematosus (SLE) and scleroderma. In some embodiments, dry eye is caused by secondary Sjogren's syndrome. Rheumatoid arthritis affects the joints and is often accompanied by dry eyes. The effects of systemic lupus erythematosus (SLE) may have in and around the eyes include: changes in the skin around the eyelids, dry eyes, inflammation of the white outer layer of the eyeball, blood vessel changes in the retina, and damage to nerves controlling eye movement and affecting vision. Approximately 20 percent of people with lupus also have secondary Sjogren's syndrome, a condition in which the tear glands do not produce sufficient tears to lubricate and nourish the eyes; the other moisture-producing glands are similarly affected. Systemic sclerosis (SSc; scleroderma) is a complex multisystem autoimmune disease of unknown etiology, characterized by vasculopathy and tissue fibrosis of the skin and various internal organs. In systemic sclerosis (SSc) patients, dry eye syndrome (DES) is the most frequent ocular feature. In some embodiments, a pharmaceutical composition comprising a therapeutically effective amount of a modified FGF-1 polypeptide of this disclosure is used to treat dry eye caused by secondary Sjogren's syndrome (SS).

In some embodiments, dry eye is age-related dry eye symptoms. Aging is a significant risk factor for dry eye. Large epidemiological studies from the Women's Health Study and Physician's Health noted that dry eye prevalence increases in women and men every five years after the age of 50, with greater prevalence in women compared to men. Anatomical and inflammation-induced age-related changes affect all components of the lacrimal gland functional unit, inclusive of lacrimal gland, conjunctiva, meibomian gland and compromise ocular surface health. In some embodiments, a pharmaceutical composition comprising a therapeutically effective amount of a modified FGF-1 polypeptide of this disclosure is used to improve function of meibomian gland and lacrimal gland and ocular surface condition.

In some embodiments, dry eye is keratoconjunctivitis sicca (KCS). Keratoconjunctivitis sicca is dryness of the conjunctiva (the membrane that lines the eyelids and covers the white of the eye) and cornea (the clear layer in front of the iris and pupil). Too few tears may be produced, or tears may evaporate too quickly. In some embodiments, a pharmaceutical composition comprising a therapeutically effective amount of a modified FGF-1 polypeptide of this disclosure is used to treat KCS by inducing adequate tear production from meibomian gland.

Allogeneic hematopoietic stem cell transplantation is a successful treatment option for hematologic malignancies. However, graft versus host disease (GVHD) is a serious complication of hematopoietic stem cell transplantation and its incidence remains high in spite of the advances in human leukocytes antigens (HLA) matching. Dry eye disease is one of the most frequent complications of ocular GVHD. The dry eye disease in ocular GVHD patients is severe, resulting in symptoms of blurred vision, photophobia, redness, gritty sensation, and pain. These symptoms cause significant visual discomfort, and reduce the overall quality of life of GVHD patients. In absence of timely and appropriate treatment, dry eye disease in GVHD patients may progress to corneal keratopathy, ulceration, and visual impairment. In some embodiments, a pharmaceutical composition comprising a therapeutically effective amount of a modified FGF-1 polypeptide of this disclosure is used to treat dry eye induced by graft versus host disease (GVHD).

Many cancer treatments, including chemotherapy, radiation, steroids and immunotherapies, are known to cause eye-related side effects such as dryness, tearing, cataracts, sensitivity to light, infection or altered vision. Among them, radiation causes damage to the lacrimal glands leading to cell damage, necrosis and apoptosis thereby releasing the inflammatory mediators which decrease the tear production and induce dry eyes. Usually, dry eye symptoms are experienced by many patients during or after treatment by radiotherapy. In some embodiments, a pharmaceutical composition comprising a therapeutically effective amount of a modified FGF-1 polypeptide of this disclosure is used to treat dry eye induced by cancer treatment comprising chemotherapy, radiation, steroids and immunotherapies. In some embodiments, a pharmaceutical composition comprising a therapeutically effective amount of a modified FGF-1 polypeptide of this disclosure is used to treat dry eye induced by radiation.

Stevens-Johnson syndrome (SJS) is life-threatening diseases of the skin and mucous membranes. After the acute-stage damage subsides, serious visual impairment and severe dry eye remains as ocular sequelae. Severe dry eye in SJS includes three important mechanisms: (1) aqueous tear deficiency, (2) decreased wettability of corneal surface, and (3) increased evaporation. In SJS patients with severe dry eye, the dryness results in immense eye pain, and unstable tear film related to dry eye result in a change/loss of vision. In some embodiments, a pharmaceutical composition comprising a therapeutically effective amount of a modified FGF-1 polypeptide of this disclosure is used to treat dry eye caused by Stevens-Johnson syndrome with suppressing chronic inflammation on the ocular surface and obtaining ocular surface stabilization.

Lacrimo-auriculo-dento-digital (LADD) syndrome is an extremely rare genetic disorder characterized by abnormalities affecting the lacrimal and salivary glands and ducts, ears, teeth and fingers and toes. The most common findings involve malformations in the network of structures of the eye that secrete tears and drain them from the eyes (lacrimal apparatus) and abnormalities of the forearms and fingers. In some embodiments, a pharmaceutical composition comprising a therapeutically effective amount of a modified FGF-1 polypeptide of this disclosure is used to treat dry eye caused by Lacrimo-auriculo-dento-digital syndrome.

Ocular cicatricial pemphigoid (OCP) is an autoimmune ocular disease that causes severe dry eye syndrome, conjunctival scarring with inferior fomix shortening and entropion along with trichiasis. In some embodiments, a pharmaceutical composition comprising a therapeutically effective amount of a modified FGF-1 polypeptide of this disclosure is used to treat dry eye caused by Ocular cicatricial pemphigoid.

Corneal abrasion causes delayed reduction in tear production. Corneal abrasions can evoke symptoms of dry eye, including changes in tear production. In some embodiments, a pharmaceutical composition comprising a therapeutically effective amount of a modified FGF-1 polypeptide of this disclosure is used to treat dry eye caused by corneal injury.

In dry eye, a chronic inflammatory reaction, possibly subclinical, is generated at the ocular surface, which can result in vital dye staining of the cornea and conjunctiva. The accumulation of inflammatory molecules at the ocular surface of dry eye patients, accompanied by a stagnant tear film and decreased level of mucins, can lead to destruction of epithelial tight junctions, and result in sloughing of the ocular surface epithelia. In some embodiments, a pharmaceutical composition comprising a therapeutically effective amount of a modified FGF-1 polypeptide of this disclosure is used to treat dry eye caused by ocular surface infection.

Riley-Day syndrome (familial dysautonomia) is an inherited condition causing autonomic nervous system dysfunction that follows an autosomal recessive inheritance pattern. Decreased lacrimation is the major ocular feature in this syndrome and it may be sufficiently severe to result in corneal damage. The blink rate is reduced, especially during crises. The lid fissures are abnormally wide contributing further to corneal drying. In some embodiments, a pharmaceutical composition comprising a therapeutically effective amount of a modified FGF-1 polypeptide of this disclosure is used to treat dry eye caused by Riley-Day syndrome.

Congenital lacrimal gland agenesis, also called congenital alacrimia, is a rare cause of dry eye and is characterized by aplasia or hypoplasia of lacrimal glands. In some embodiments, a pharmaceutical composition comprising a therapeutically effective amount of a modified FGF-1 polypeptide of this disclosure is used to treat dry eye caused by congenital alacrimia.

Among many etiological factors, an association of Vitamin D deficiency with dry eyes and tear film insufficiency has received attention in recent studies. Vitamin D reduces tear osmolarity and improves the stability of tear film. Lacrimal, salivary, and parotid gland functions may be directly affected by vitamin D. Vitamin A deficiency can also lead to corneal dryness (xerosis), keratomalacia, and corneal ulceration. Dry eye can be caused directly or indirectly by deficiency of nutrition including, but not limited to, lutein, zeaxanthin, vitamin C, vitamin E, Omega-3 fatty acids EPA and DHA, and zinc. In some embodiments, a pharmaceutical composition comprising a therapeutically effective amount of a modified FGF-1 polypeptide of this disclosure is used to treat dry eye caused by nutritional disorders or deficiencies.

In some embodiments, a pharmaceutical composition comprising a therapeutically effective amount of a modified FGF-1 polypeptide of this disclosure is administered to treat dry eye caused by pharmacologic side effects after administering medications comprising antidepressants, antihistamines, topical retinoid-antibiotic combination, common acne control medication, birth control, painkillers, beta blockers, gastrointestinal medications, antipsychotic drugs, hormone replacements, and chemotherapy medications.

Patients with Parkinson's disease suffer from dry eye due to rare blinking, reduced tear production and altered composition of tear fluid. Diabetic retinopathy is a disease of the retina and one of the most common causes of blindness in people between 20 and 65. In particular, due to restricted blood circulation in the structures of the eye, the nerves, which supply the eye, can be damaged. The nerves affected can be those which control the lacrimal glands. In this case, tear production is no longer adequately controlled, too little lacrimal fluid is produced, and the eyes dry out. The thyroid gland is a central control point for metabolic processes. An underactive thyroid gland distributes too little of the hormones thyroxine (T4) and triiodothyronine (T3). The whole metabolism of the body is slowed down by the lack of hormones, which brings with it a creeping development of symptoms. Additionally, the skin can often appear rough and dry, and the eyes can be dry out. In some embodiments, a pharmaceutical composition comprising a therapeutically effective amount of a modified FGF-1 polypeptide of this disclosure is used to treat dry eye caused by a systemic disease comprising Parkinson's disease, diabetes or thyroid disease.

Viruses can trigger autoimmune reactions and those events could eventually lead to overproduction of immunoglobulins, autoantibodies and memory lymphocytes and subsequently tissue damage and dysfunction due to apoptosis and inflammation and may result in the occurrence of Sjogren syndrome-like illness. In some embodiments, a pharmaceutical composition comprising a therapeutically effective amount of a modified FGF-1 polypeptide of this disclosure is administered to treat dry eyes caused by bacterial infections. Bacteria can also infect the lacrimal glands. These include, for example, the pathogens for scarlet fever, tuberculosis or syphilis. In some embodiments, a pharmaceutical composition comprising a therapeutically effective amount of a modified FGF-1 polypeptide of this disclosure is used to treat dry eye caused by virus infection or bacterial infection. In some embodiments, the virus comprises human T-cell lymphotropic virus (HTLV), human immunodeficiency virus (HIV), Epstein-Barr virus (EBV), and hepatitis C virus (HCV).

Rosacea is a common skin disease that causes rosy patches on your skin and dry or oily spots, usually on the face. There's a related condition called ocular rosacea that can cause dry eye. Pruritus is the medical term for itching. Ocular itching may be linked with dry eye. It's triggered by the same things that set off itching elsewhere on the body, such as allergic reactions to medications or cosmetic products. In some embodiments, a pharmaceutical composition comprising a therapeutically effective amount of a modified FGF-1 polypeptide of this disclosure is used to treat dry eye caused by skin disease comprising rosacea, blepharitis, or pruritus on the eyelids or near the eye.

Eye contact with either air pollutants or adverse indoor and/or outdoor environmental conditions can affect tear film composition and ocular surface components. About 40% of soft contact lens wearers report the sensation of dry eye and 25% suffers from moderate to severe symptoms. Use of computers and display devices with a screen decreases the number of eye blinks, leading to incomplete blinking, evaporation of tears, and subsequently to dry eye disease. In some embodiments, a pharmaceutical composition comprising a therapeutically effective amount of a modified FGF-1 polypeptide of this disclosure is used to treat dry eye caused by environmental exposure by suppressing dry eye symptoms.

The research that was conducted on dry eye in visual display terminal (VDT) workers showed decreased tear secretion in office workers, which was dependent on the number of years of VDT use. Next, they demonstrated lacrimal gland dysfunction as a decrease in tear secretion, which recovered after cessation of the swing activity. Provided herein in some embodiments is a method of treating dry eye caused by the exposure to visual display terminal (e.g., computer or television), by administering a pharmaceutical composition comprising a therapeutically effective amount of a modified FGF-1 polypeptide described herein.

Damage to the corneal afferent nerves during PRK and LASIK disrupts sensory input into the ocular surface lacrimal gland feedback system. In some embodiments, a pharmaceutical composition comprising a therapeutically effective amount of a modified FGF-1 polypeptide of this disclosure is used to treat dry eye occurred as a side effect of laser assisted vision correction procedures comprising photorefractive keratectomy (PRK), laser-assisted sub-epithelial keratectomy (LASEK) and laser-assisted in situ keratomileusis (LASIK) by suppressing dry eyes symptoms.

In some embodiments, a pharmaceutical composition comprising a therapeutically effective amount of a modified FGF-1 polypeptide of this disclosure or a pharmaceutical composition comprising the same is administered to reduce an elevated expression of any one or more biomarkers selected from IL-6, TNF-alpha, IL-1beta, IL-12, IFN-gamma, IL-17A, IL-17F, IL-22, IL-4 and IL-5, IL-10, IL-13, TGF-beta, IL-1RA, IL8/CXCL8, IP-10/CXCL 10, MMP-9, VEGF, EGF, Latoferrin, MIP-1alpha/CCL3, MIP-1beta/CCL4, RANTES/CCL5, Fractalkine/CX3CL1, CXCL9, CXCL10, CXCL11, MCP-1/CCL2, LPRR4, LPRR3, nasopharyngeal carcinoma associated PRP4 and α-1 antitrypsin, PIP (prolactin-inducible protein), LCN-1 (Lipocalin-1), α-enolase, S100A8/Calgranulin A, S100A9/Calgranulin B, S100A4 and S100A1, Annexin A1 (ANXA1), Annexin A11 (ANXA11), MUC5AC, Cathepsin S, Neuromediators (e.g., substance P, NGF, VIP, and CGRP), HLA-DR (dendritic cell maturation marker), Fas (CD95, apoptosis-related marker), CD40 (costimulatory protein on antigen-presenting cell [APC]), IFN-γ, and TNF-α, Keratin 10 (Krt10)/Krt1, Krt16/Krt6, Krt17, C3, S100A6, S100A8, CP, APOD, ORM2, ANXA1, CLU, ORM1, LPO, or other potential biomarkers (e.g., IgE, Tryptase, Histamine, and ECP).

### Pharmaceutical Compositions, Methods of Administration, and Dosing

Pharmaceutical compositions comprising a modified FGF-polypeptide as described herein, for use in methods of this disclosure, in some embodiments, is formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, in some instances, refers to a mixture of a modified FGF-1 polypeptides with other chemical components, such as carriers, stabilizers, diluents, dispersing agents, suspending agents, thickening agents, and/or excipients, and, optionally, other therapeutic and/or prophylactic ingredients. The pharmaceutical composition facilitates administration of the modified FGF to an organism. In practicing the methods of treatment or use provided herein, therapeutically effective amounts of modified FGF-1 polypeptides described herein are administered in a pharmaceutical composition to a mammal having an ocular disease, disorder, or condition to be treated. In some embodiments, the mammal is a human. A therapeutically effective amount can vary widely depending on the severity of the disease, the age and relative health of the subject, the potency of the compound used and other factors. A pharmaceutically acceptable or suitable composition includes an ophthalmologically suitable or acceptable composition.

A pharmaceutical composition (e.g., for delivery by injection or for application as an eye drop) in some embodiments is in the form of a liquid or solid. A liquid pharmaceutical composition includes, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. Physiological saline is commonly used as an excipient, and an injectable pharmaceutical composition or a composition that is delivered ocularly (for example, as an eye drop) is preferably sterile.

A modified FGF-polypeptide or pharmaceutical composition described herein can be delivered to a subject by any suitable means, including, for example, topically, intraocularly, intracamerally, orally, parenterally, intravenously, intraperitoneally, intranasally (or other delivery methods to the mucous membranes, for example, of the nose, throat, and bronchial tubes), or by local administration to the eye, or by an intraocular or periocular device. Modes of local administration can include, for example, topical application, eye drops, intraocular injection or periocular injection. Periocular injection typically involves injection of the compound under the conjunctiva or into the Tennon's space (beneath the fibrous tissue overlying the eye). Intraocular injection typically involves injection of the modified FGF or pharmaceutical composition into the vitreous. In certain embodiments, the administration is non-invasive, such as by topical application or eye drops. In some embodiments, the administration is via a combination of topical and intracameral method.

A modified FGF-1 or pharmaceutical composition described herein in some embodiments is formulated for administration using pharmaceutically acceptable (suitable) carriers or vehicles as well as techniques routinely used in the art. A pharmaceutically acceptable or suitable carrier includes an ophthalmologically suitable or acceptable carrier. A carrier is selected according to the solubility of the modified FGF-1. Suitable ophthalmological compositions and formulations include those that are administrable locally to the eye, such as by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

For injection, the modified FGF-1 or pharmaceutical composition in some embodiments is provided in an injection grade saline solution, in the form of an injectable liposome solution, slow-release polymer system or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

In some embodiments, the modified FGF-1 polypeptide or pharmaceutical composition (e.g., an ophthalmic formulation) is administered via microneedles into the cornea (Jiang et al. (2007). Invest Ophthalmol Vis Sci 48(9): 4038-4043). A microneedle array is coated with the modified FGF or pharmaceutical composition and pressed against the cornea such that the microneedles penetrate into the corneal stroma but do not penetrate the entire cornea. It is then removed, and the modified FGF or pharmaceutical composition is left behind in the corneal stroma. This modified FGF or pharmaceutical composition can stimulate the corneal cells to proliferate and migrate, and suppresses the scarring response that the stromal cells normally have.

In some embodiments, the composition is formulated for intraocular delivery. Intraocular delivery comprises intravitreal delivery, corneal injections, intracameral delivery. In some embodiment the composition is formulated for intracameral delivery. In some embodiments the composition is formulated for intravitreal delivery. The formulation is an injectable liquid, may comprise a very small volume, and the density of the injectable liquid formulation may be adjusted such its release in the targeted space does not incur injury to the tissue. In some embodiments, the volume for intracameral delivery is less than about 20 microliters, less than about 10 microliters, less than about 5 microliters, less than about 2.5 microliters, or about 1 microliter. Provided herein is a formulation for intraocular delivery, comprising: a modified FGF-1 polypeptide comprising an amino acid sequence set forth in SEQ ID NO: 1, or having an amino acid sequence that has at least 90% sequence identity to SEQ ID NO: 1, and comprising at least 1, 2, 3, 4 or 5 single amino acid mutations; and L-methionine. The modified FGF-1 polypeptide in the formulation in some embodiments is present at greater than about 95% pure and the polypeptide is in monomeric form in the formulation. In some embodiments, the polypeptide further comprises an extension peptide positioned between the N-terminal methionine residue and the first residue of SEQ ID NO: 1. In some embodiments, the formulation comprises a modified FGF-1 comprising an amino acid sequence set forth in any one of the following sequences, SEQ ID NOs: 2, 205, 206, 3-8, 14-18, 24-28, 93, 94, 96-117, 118-141, 207, 146-149, and 174-204, or a sequence that has at least 90% sequence identity to the sequences, or is a functional fragment thereof.

In some embodiments, an formulation is hereby provided, the formulation comprises a required dose and concentration of the modified FGF-1, and an excipient, comprising one or more of sodium chloride; ammonium sulfate; monobasic potassium phosphate; dibasic sodium phosphate dihydrate; ethylenediaminetetraacetic; and L-Methionine. In some embodiments, the formulation comprises: a modified FGF-1 polypeptide; at least about 50 mM dibasic sodium phosphate dihydrate; at least about 100 mM sodium chloride; at least about 10 mM ammonium sulfate; at least about 0.1 mM ethylenediaminetetraacetic acid (EDTA); at least about 5 mM L-Methionine; and at least about 0.01% polysorbate 80 (w/v). The formulation comprising the modified FGF-1 polypeptide comprises one or more mutations selected from the group consisting of: Cys16Ser, Ala66Cys, and Cys117Val, Lys12Val, Cys16Ser, Ala66Cys, Cys117Val, and Pro134Val, and wherein the modified FGF-1 polypeptide further comprises at least one residue of the peptide ALTEK. In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1, and at least one residue of the peptide ALTEK located between the N-terminal methionine and position 1 of SEQ ID NO: 1. In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1. In some embodiments, the modified FGF-1 polypeptide comprises one or more mutations comprising the following mutations of SEQ ID NO: 1: Cys16Ser, Ala66Cys, and Cys117Val.

In some embodiments, the formulation comprising a required dose and concentration of the modified FGF-1, and an excipient, comprising one or more of sodium chloride; ammonium sulfate; monobasic potassium phosphate; dibasic sodium phosphate dihydrate; ethylenediaminetetraacetic; and L-Methionine as disclosed herein is suitable for delivery by injection. In some embodiments, the formulation comprising a modified FGF-1 polypeptide; at least about 50 mM dibasic sodium phosphate dihydrate; at least about 100 mM sodium chloride; at least about 10 mM ammonium sulfate; at least about 0.1 mM ethylenediaminetetraacetic acid (EDTA); at least about 5 mM L-Methionine; and at least about 0.01% polysorbate 80 (w/v) as disclosed herein is suitable for delivery by injection. In some embodiments, the formulation comprising a modified FGF-1 polypeptide comprising one or more mutations selected from the group consisting of: Cys16Ser, Ala66Cys, and Cys117Val, Lys12Val, Cys16Ser, Ala66Cys, Cys117Val, and Pro134Val, and wherein the modified FGF-1 polypeptide further comprising at least one residue of the peptide ALTEK as disclosed herein is suitable for delivery by injection. In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1, and at least one residue of the peptide ALTEK located between the N-terminal methionine and position 1 of SEQ ID NO: 1. In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1. In some embodiments, the modified FGF-1 polypeptide comprises one or more mutations comprising the following mutations of SEQ ID NO: 1: Cys16Ser, Ala66Cys, and Cys117Val.

In some embodiments, the formulation or the pharmaceutically suitable excipient therein comprises human serum albumin (HSA) and/or polysorbate 80. In some embodiments, the formulation comprises L-Methionine. In some embodiments, the L-Methionine is present at a concentration between 1 mM to 20 mM in the formulation. In some embodiments, the L-Methionine is present at a concentration between 2 mM to 10 mM in the formulation. In some embodiments, the L-Methionine is present at a concentration between 1 mM to 10 mM in the formulation. In some embodiments, the L-Methionine is present at a concentration between 2.5 mM to 15 mM in the formulation. In some embodiments, the L-Methionine is present at a concentration of about 5 mM in the formulation. For delivery of a composition comprising at least one of the modified FGF-1 polypeptides described herein via a mucosal route, which includes delivery to the nasal passages, throat, and airways, the composition may be delivered in the form of an aerosol. The compound in some embodiments is in a liquid or powder form for intramucosal delivery. For example, the composition may be delivered via a pressurized aerosol container with a suitable propellant, such as a hydrocarbon propellant (e.g., propane, butane, isobutene). The composition in some embodiments is delivered via a non-pressurized delivery system such as a nebulizer or atomizer.

For delivery of a composition comprising at least one of the modified FGF-1 polypeptides described herein via a mucosal route, which includes delivery to the nasal passages, throat, and airways, the composition may be delivered in the form of an aerosol. The compound in some embodiments is in a liquid or powder form for intramucosal delivery. For example, the composition may be delivered via a pressurized aerosol container with a suitable propellant, such as a hydrocarbon propellant (e.g., propane, butane, isobutene). The composition may be delivered via a non-pressurized delivery system such as a nebulizer or atomizer.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

The modified FGF-1 polypeptides or pharmaceutical compositions described herein may be formulated for sustained or slow-release. Such compositions may generally be prepared using well known technology and administered by, for example, periocular, intraocular, rectal, oral or subcutaneous implantation, or by implantation at the desired target site, or by topical application. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained-release formulation depends upon the site of implantation, the rate and expected duration of release, and the nature of the condition to be treated or prevented.

In some embodiments, methods of this disclosure comprise administering a composition comprising a modified FGF-1 polypeptide, citrate or histidine, sorbitol, and polysorbate. The concentrations, in some embodiments, are about 1 mM citrate or Histidine, about 5% sorbitol, about 0.1% polysorbate 80, and the formulation has a pH of about 5.8. In some embodiments, the concentration of citrate or Histidine comprises 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, and 4 mM, for example from about 0.1 mM to about 4 mM, from about 0.2 mM to about 3.5 mM, from about 0.3 mM to about 3 mM, from about 0.4 mM to about 2.5 mM, from about 0.6 mM to about 2 mM, 0.8 mM to about 1.5 mM, and from about 0.9 mM to about 1 mM. In some embodiments, the pH of the formulation is about 4.8, 5.0, 5.2, 5.4, 5.6, 5.8, 6.0, 6.2, 6.4, 6.6, 6.8, or 7.0, for example from about 4.0 to about 8.0, from about 4.5 to about 7.2, from about 5 to about 7.0, from about 5.5 to about 7.2, and from about 4.8 to about 6.5. In some embodiments, the concentration of sorbitol comprises 3.8%, 4.0%, 4.2%, 4.4%, 4.6%, 4.8%, 5.0%, 5.2%, 5.4%, 5.6%, 5.8%, 6.0%, and 6.2%, for example from about 3.6% to about 6.5%, from about 4.0% to about 6.0%, from about 4.5% to about 6.5%, from about 5.0% to about 6.2%, and from about 5.5% to about 6.5%. In some embodiments, the concentration of polysorbate 80 comprises 0.02%, 0.04%, 0.06%, 0.08%, 0.10%, 0.12%, 0.14%, 0.16%, 0.18%, 0.2%, and 0.22%, for example from about 0.02% to about 0.2%, from about 0.03% to about 0.9%, from about 0.04% to about 0.18%, from about 0.06% to about 0.15%, and from about 0.08% to about 0.14%.

In some embodiments, methods of this disclosure comprise administering a composition comprising a modified FGF-1 polypeptide, sodium chloride, ammonium sulfate, and di-sodium hydrogen phosphate. The concentrations, in some embodiments, are 3 mg/mL of the modified FGF-1 polypeptide, about 800 mM of sodium chloride, about 320 mM of ammonium sulfate, about 20 mM of di-sodium hydrogen phosphate, and has a pH of about 7.4. In some embodiments, the concentration of the modified FGF-1 comprises 0.5 mg/mL, 1 mg/mL, 1.5 mg/mL, 2 mg/mL, 2.5 mg/mL, 3 mg/mL, 3.5 mg/mL, 4 mg/mL, 4.5 mg/mL, and 5 mg/mL, for example from 0.5 mg/mL to 5 mg/mL, from 1 mg/mL to 4.5 mg/mL, from 1.5 mg/mL to 4 mg/mL, from 2 mg/mL to 3.5 mg/mL, and from 2.5 mg/mL to 3 mg/mL. In some embodiments, the concentration of sodium chloride comprises about 500 mM, about 550 mM, about 600 mM, about 650 mM, about 700 mM, about 750 mM, about 800 mM, about 850 mM, about 900 mM, and about 950 mM, for example from about 500 mM to about 950 mM, from about 550 mM to about 900 mM, from about 600 mM to about 850 mM, from about 650 mM to about 800 mM, and from about 700 mM to about 750 mM. The concentration of ammonium sulfate comprises about 260 mM, about 280 mM, about 300 mM, about 320 mM, about 340 mM, about 360 mM, about 380 mM, and about 400 mM, for example from about 260 mM to about 400 mM, from about 280 mM to about 380 mM, from about 300 mM to about 360 mM, and from about 320 mM to about 340 mM. The concentration of di-sodium hydrogen phosphate comprises about 10 mM, about 15 mM, about 20 mM, about 25 mM, about 30 mM, about 35 mM, about 40 mM, about 45 mM, and about 50 mM, for example from about 10 mM to about 50 mM, from about 15 mM to about 45 mM, from about 20 mM to about 40 mM, and from about 25 mM to about 35 mM. In some embodiments, the pH of the formulation is about 5.6, 5.8, 6.0, 6.2, 6.4, 6.6, 6.8, 7.0, 7.2, 7.4, 7.6, 7.8, 8.0, 8.2, 8.4, and 8.6 for example from about 5.6 to about 8.6, from about 5.8 to about 8.4, from about 6 to about 8.2, from about 6.2 to about 8.0, from about 6.4 to about 7.8, from about 6.6 to about 7.6, from about 6.8 to about 7.4, and from about 7.0 to about 7.2.

In some embodiments, methods of this disclosure comprise administering a composition comprising a modified FGF-1 polypeptide at a dose of about 10 ng-1000 ng per eye per administration, wherein the administration is intracameral. In some embodiments, a modified FGF-1 polypeptide or pharmaceutical compositions described herein is administered at a concentration of about 0.1 to 10 μg/ml, wherein the administration is topical. In some embodiments, a modified FGF-1 polypeptide or pharmaceutical compositions described herein is administered at a dose of about 0.3 mg/kg to about 10 mg/kg. In some embodiments, a modified FGF-1 polypeptide or pharmaceutical compositions described herein is administered at a dose of about 0.3 μg/eye to about 3 μg/eye per administration.

In some embodiments, methods of this disclosure comprise administering a composition comprising a modified FGF-1 polypeptide about one to five times a day. In some embodiments, a modified FGF-1 polypeptide or pharmaceutical compositions described herein is administered about two times a day. In some embodiments, a modified FGF-1 polypeptide or pharmaceutical composition described herein is administered about three times a day. In some embodiments, a modified FGF-1 polypeptide or pharmaceutical composition described herein is administered for at least five consecutive days. In some embodiments, a modified FGF-1 polypeptide or pharmaceutical composition described herein is administered for at least seven consecutive days. In some embodiments, a modified FGF-1 polypeptide or pharmaceutical compositions described herein is administered at least for 15 days, 21 days, 24 days, 28 days, 30 days. In some embodiments, a modified FGF-1 polypeptide or pharmaceutical compositions described herein is administered via intracameral or intravitreal injection every 7 to 30 days.

Systemic drug absorption of a drug or composition administered via an ocular route is known to those skilled in the art (see, e.g., Lee et al., Int. J Pharm. 233:1-18 (2002)). In one embodiment, a compound described herein is delivered by a topical ocular delivery method (see, e.g., Curr. Drug Metab. 4:213-22 (2003)). The composition may be in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used.

In another embodiment, the modified FGF solution or pharmaceutical composition (e.g., an ophthalmic formulation) contains hyaluronic acid, carboxymethyl cellulose, or other polysaccharides that provide increased ocular tolerability, viscosity and osmolality to produce a comfortable ocular solution.

The dose of the modified FGF or pharmaceutical composition comprising at least one of the modified FGF-1 polypeptides described herein may differ, depending upon the patient's (e.g., human) condition, that is, stage of the ocular disease, disorder, or condition, general health status, age, and other factors that a person skilled in the medical art will use to determine dose. When the composition is used as eye drops, for example, one to several drops per unit dose, preferably 1 or 2 drops (about 50 μl per 1 drop), may be applied about 1 to about 6 times daily.

Pharmaceutical compositions may be administered in a manner appropriate to the disease, disorder, or condition to be treated (or prevented) as determined by persons skilled in the medical arts. An appropriate dose and a suitable duration and frequency of administration will be determined by such factors as the condition of the patient, the type and severity of the patient's disease, disorder, or condition, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free, or a lessening of symptom severity). For prophylactic use, a dose should be sufficient to prevent, delay the onset of, or diminish the severity of an ocular disease, disorder, or condition. Optimal doses may generally be determined using experimental models and/or clinical trials. The optimal dose may depend upon the body mass, weight, or blood volume of the patient.

In various embodiments, a modified FGF-1 polypeptide of the present disclosure may be administered as a daily dose over a period of time to a subject. The doses of the modified FGF-1 polypeptides or pharmaceutical compositions can be suitably selected depending on the clinical status, condition and age of the subject, dosage form and the like. In some embodiments, a modified FGF-1 polypeptide of the present disclosure may be administered chronically or long-term. In some embodiments, a modified FGF-1 polypeptide of the present disclosure may be administered for a period of days, weeks, months, years or continued therapy over the lifetime of a subject. In some embodiments, a modified FGF-1 polypeptide of the present disclosure may be administered for a period of about 7 days, 15 days, about 21 days, about 30 days, about 3 months, about 6 months, about 12 months, about 18 months, about 2 years, about 5 years, about 7 years, about 10 years, about 15 years, about 20 years, about 25 years, about 30 years, about 35 years, or about 40 years. In some embodiments, a treatment regime may be determined for an individual subject dependent on various factors. In some embodiments, the treatment regimen is about 2 weeks for an acute exposure and several months to a year for a long term exposure. In some embodiments, the treatment regimen is chronic.

At least one modified FGF described herein can be administered to human or other nonhuman vertebrates, for use in a method as provided herein. In certain embodiments, the modified FGF is substantially pure, in that it contains less than about 5% or less than about 1%, or less than about 0.1%, of other organic molecules, such as contaminating intermediates or by-products that are created, for example, in one or more of the steps of a synthesis method. In other embodiments, a combination of one or more modified FGF-1 polypeptides described herein can be administered.

The compositions described herein can be administered for prophylactic and/or therapeutic treatments. In therapeutic applications, the compositions are administered to a patient already suffering from a disease or condition (e.g., dry eye diseases including Meibomian Gland Dysfunction and Sjogren's Syndrome), in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. In prophylactic applications, compositions described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. In the case wherein the patient's condition does not improve, upon the doctor's discretion the administration of the compositions may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compositions may be given continuously; alternatively, the dose of drug being administered may be temporarily reduced or temporarily suspended for a certain length of time (i.e., a “drug holiday”). Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms. The desired dose may conveniently be presented in a single dose or as divided doses administered simultaneously (or over a short period of time) or at appropriate intervals, for example as two, three, four or more sub-doses per day.

The pharmaceutical compositions described herein may be in unit dosage forms suitable for single administration of precise dosages. In unit dosage form, the formulation is divided into unit doses containing appropriate quantities of one or more modified FGF-1 polypeptides. The unit dosage may be in the form of a package containing discrete quantities of the formulation. Non-limiting examples are packaged tablets or capsules, and powders in vials or ampoules. Aqueous suspension or solution compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

The modified FGF-1 polypeptides and pharmaceutical compositions may also be used in combination with other therapeutic agents that are selected for their therapeutic value for the condition to be treated. The modified FGF-1 polypeptides and pharmaceutical compositions may also be used in combination with other therapeutic agents that are selected for their therapeutic value for treating dry eye symptoms. Such agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the clinician. The initial administration can be made according to established protocols recognized in the field, and then, based upon the observed effects, the dosage, modes of administration and times of administration can be modified by the clinician.

The particular choice of these optional additional agents used will depend upon the diagnosis of the attending physicians and their judgment of the condition of the patient and the appropriate treatment protocol. The agents may be administered concurrently (e.g., simultaneously, essentially simultaneously or within the same treatment protocol) or sequentially, depending upon the nature of the disease, disorder, or condition, the condition of the patient, and the actual choice of agents used. The determination of the order of administration, and the number of repetitions of administration of each therapeutic agent during a treatment protocol, is well within the knowledge of the physician after evaluation of the disease being treated and the condition of the patient.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In some embodiments, the therapeutic agent comprises a profibrotic factor antagonist that targets a profibrotic factor. As used herein the term “profibrotic factors” refers to cytokines, growth factors or chemokines which have been observed to promote the accumulation of fibroblasts and deposition of collagen in various tissues. A number of cytokines and growth factors have been reported to be involved in regulating tissue remodeling and fibrosis. These include the “profibrotic cytokines” such as transforming growth factor beta (TGF-β), interleukin-4 (IL-4), interleukin-5 (IL-5), and interleukin-13 (IL-13), which have been shown to stimulate collagen synthesis and fibrosis in fibrotic tissues (Letterio et al. Ann Rev. Immunol. 16, 137-161 (1998), Fertin et al., Cell MoI. Biol. 37, 823-829 (1991), Doucet et al., J. Clin. Invest. 101, 2129-2139 (1998). Interleukin-9 (IL-9) has been shown to induce airway fibrosis in the lungs of mice (Zhu et al., J. Clin. Invest. 103, 779-788(1999)). In addition to TGF-β, other cytokines or growth factors which have been reported to increase fibrosis in the fibrotic disorder idiopathic pulmonary fibrosis (IPF) include granulocyte/macrophage-colony stimulating factor (GM-CSF), tumor necrosis factor alpha (TNF-α), interleukin-1 beta (IL-10), and connective tissue growth factor (CTGF) (Kelly et al. Curr Pharmaceutical Pes 9: 39-49 (2003)). Cytokines and growth factors reported to be involved in promoting pulmonary fibrosis occurring in scleroderma include TGF-β, interleukin-1 beta (IL-1β), interleukin-6 (IL-6), oncostatin M (OSM), platelet derived growth factor (PDGF), the type 2 cytokines IL-4 and IL-13, IL-9, monocyte chemotactic protein 1 (CCL2/MCP-1), and pulmonary and activation-regulated chemokine (CCL1 8/P ARC) (Atamas et al., Cyto Growth Fact Rev 14: 537-550 (2003)).

It has been suggested that proinflammatory cytokines in tears may have a key role in the pathogenesis of several corneal diseases, including dry eye disease, as was found in keratoconus, GVHD, conjunctivitis, as well as in the development of corneal neovascularization (NV). Increased levels of proinflammatory cytokines, such as IL-1, IL-6, and IL-8, and decreased epidermal growth factor (EGF) levels have been reported in eyes with Sjogren's\ddot{ } syndrome. It was also showed that the tear cytokine levels are strongly correlated with dry-eye-related clinical parameters. Hyperosmolar stress also has a direct proinflammatory effect on the ocular surface that increases the tear cytokine levels. A correlation between corneal DC density and tear inflammatory cytokines in dry eye with rheumatoid arthritis (RA) has been reported. In addition, it has been found that IL-1 and IL-6 concentrations decreased after the systemic treatment of RA. Reduced tear cytokine levels has been also reported as an inflammatory biomarker for the effectiveness of topical steroids or intense pulsed light in treating dry eye due to MGD. In these reports, inflammatory cytokine levels correlated well with ocular surface parameters.

Profibrotic factors that may be targeted with profibrotic factor antagonists as part of the combination therapy with a modified FGF-1 polypeptide of this disclosure, include without limitation, a growth factor type β (TGF-β, including TGF-β1-5), VEGF, EGF, PDGF, IGF, RANTES, members of the interleukin family (e.g., IL-I, IL-4, IL-5, IL-6, IL-8 and IL-13), tumor necrosis factor type alpha (TNF-α), platelet-derived growth factor (PDGF), basic fibroblast growth factor (bFGF), monocyte chemoattractant protein type 1 (MCP-I), macrophage inflammatory protein (e.g., MIP-1α, MIP-2), connective tissue growth factor (CTGF), endothelin-1, angiotensin-II, leptin, chemokines (e.g., CCL2, CCL12, CXCL12, CXCR4, CCR3, CCR5, CCR7, SLC/CCL21), integrals (e.g., αlβl, β2βl αvβó, αvβ3), tissue inhibitors of matrix metalloproteinases (e.g., TIMP-I, TIMP-2) and other factors known to promote or be related to the formation, growth, or maintenance of fibrotic tissue.

In some embodiments, the modified FGF-1 polypeptide is incorporated into formulations that contain other active ingredients such as steroids, antibiotics, anti-inflammatories, cytokines such as IL-1 or analogs of IL-1, or antagonists of cytokines such as inhibitors of IL-17.

Other exemplary cytokines include, but are not limited to, interleukins (e.g., IL-1, IL-2, IL-3, IL-4, IL-5, IL-6, IL-7, IL-8, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), IFN family members (IFN-1, IFN-γ, and IFN-III) and migration inhibitory factor MIF.

In some embodiments, combinations or pharmaceutical compositions described herein are administered in immunosuppressive therapy to reduce, inhibit, or prevent activity of the immune system. Immunosuppressive therapy is clinically used to: prevent the rejection of transplanted organs and tissues; treatment of autoimmune diseases or diseases that are most likely of autoimmune origin; and treatment of some other non-autoimmune inflammatory diseases.

In some embodiments, a pharmaceutical composition comprising a therapeutically effective amount of a modified FGF-1 polypeptide described herein are administered with one or more anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, and COX-2 specific inhibitors (such as, but not limited to, celecoxib, rofecoxib, valdecoxib, parecoxib, etoricoxib, lumiracoxib, CS-502, JTE-522, L-745,337 and NS398).

Corticosteroids, include, but are not limited to: betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Other agents used as anti-inflammatories include those disclosed in U.S. patent publication 2005/0227929, herein incorporated by reference.

Some commercially available anti-inflammatories include, but are not limited to: Arthrotec® (diclofenac and misoprostol), Asacol® (5-aminosalicyclic acid), Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer® (aspirin), Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Advil®, Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In one embodiment, compositions described herein are administered with leukotriene receptor antagonists including, but are not limited to, BAY u9773 (see EP 00791576; published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

In some embodiments, a pharmaceutical composition comprising a therapeutically effective amount of a modified FGF-1 polypeptide is administered in combination with topical therapy for dry eye symptoms.

In some embodiments, a pharmaceutical composition comprising a therapeutically effective amount of a modified FGF-1 polypeptide is administered in combination with lifitegrast for the treatment of dry eye, which inhibit T-cell activation and cytokine production.

In some embodiments, a pharmaceutical composition comprising a therapeutically effective amount of a modified FGF-1 polypeptide is administered in combination with injections of steroid or mitomycin C to slow progression of dry eye symptoms.

In some embodiments, a pharmaceutical composition comprising a therapeutically effective amount of a modified FGF-1 polypeptide is administered in combination with topical cyclosporine and tacrolimus ointment to aid in the control of ocular surface inflammation.

In some embodiments, a pharmaceutical composition comprising a therapeutically effective amount of a modified FGF-1 polypeptide is administered in combination with Dapsone as the first-line treatment for mild to moderate dry eye disease.

In some embodiments, a pharmaceutical composition comprising a therapeutically effective amount of a modified FGF-1 polypeptide is administered in combination with systemic corticosteroid pulse and concurrent immunosuppressant therapy with azathioprine, mycophenolate mofetil, methotrexate, or cyclosporine for moderate to severe dry eye symptoms or lack of response to Dapsone or other first-line alternatives.

In some embodiments, a pharmaceutical composition comprising a therapeutically effective amount of a modified FGF-1 polypeptide is administered in combination with orbital radiation therapy.

In some embodiments, a pharmaceutical composition comprising a therapeutically effective amount of a modified FGF-1 polypeptide is administered in combination with biologics such as etanercept or rituximab and intravenous immunoglobulin therapy that are reserved for patients who have a poor response to conventional therapy.

In some embodiments, a pharmaceutical composition comprising a therapeutically effective amount of a modified FGF-1 polypeptide is administered in combination with cataract surgery to reduce chances of increased conjunctival inflammation, rapid progression of keratopathy, and conjunctival scarring after the surgery.

In some embodiments, a pharmaceutical composition comprising a therapeutically effective amount of a modified FGF-1 polypeptide is administered in combination with B-cell-targeted therapies for dry eye associated with Sjogren's syndrome. In some embodiments, the B-cell-targeted therapy comprises administering to a subject in need rituximab, epratuzumab, belimumab, ianalumab (VAY736), or baminercept.

In some embodiments, a pharmaceutical composition comprising a therapeutically effective amount of a modified FGF-1 polypeptide is administered in combination with CD20-targeted therapies, CD22 (cluster of differentiation-22)-targeted therapies, BAFF and APRIL-targeted therapies, lymphotoxin beta receptor (LTPR)-targeted therapies, or T cell-targeted therapies for dry eye associated with Sjogren's syndrome. In some embodiments, the T cell-targeted therapy comprises administering to a subject in need abatacept or alefacept.

In some embodiments, a pharmaceutical composition comprising a therapeutically effective amount of a modified FGF-1 polypeptide is administered in combination with Mesenchyme stem cell transplantation for dry eye associated with Sjogren's syndrome.

In some embodiments, a pharmaceutical composition comprising a therapeutically effective amount of a modified FGF-1 polypeptide is administered in combination with gene therapy by engineering cells in way to produce therapeutic protein locally.

In some embodiments, a pharmaceutical composition comprising a therapeutically effective amount of a modified FGF-1 polypeptide is administered in combination with intraductal meibomian gland probing to relieve dry eye symptoms associated with MGD.

In some embodiments, a pharmaceutical composition comprising a therapeutically effective amount of a modified FGF-1 polypeptide is administered in combination with emulsion eye drops containing lipids as optional treatment for dry eye associated with MGD.

In some embodiments, a pharmaceutical composition comprising a therapeutically effective amount of a modified FGF-1 polypeptide is administered in combination with LipiFlow® treatment (TearScience®, Morrisville, NC, USA), which could apply heat to both the upper and lower palpebral conjunctival surfaces in addition to pressure to the external eyelid at the same time to express the meibomian gland for treatment of dry eye associated with MGD.

N-acetyl-cysteine (NAC) is an acetylated derivative of the natural amino acid, l-cysteine. It has mucolytic, anti-collagenolytic, and antioxidant properties. It also modulates the cellular redox status to influence several inflammatory pathways, leading to decreased nuclear factor-kappa B activity, which regulates several proinflammatory genes that regulate the inflammation pathways. In some embodiments, a pharmaceutical composition comprising a therapeutically effective amount of a modified FGF-1 polypeptide is administered in combination with N-acetyl-cysteine (NAC) for treatment of dry eye associated with MGD.

Topical azithromycin has been shown to be a potentially effective and well tolerated treatment for meibomian gland dysfunction. In some embodiments, a pharmaceutical composition comprising a therapeutically effective amount of a modified FGF-1 polypeptide is administered in combination with topical azithromycin for treatment of dry eye associated with MGD.

In some embodiments, a pharmaceutical composition comprising a therapeutically effective amount of a modified FGF-1 polypeptide is administered in combination with oral supplementation with omega-3 essential fatty acids for treatment of dry eye associated with MGD.

In some embodiments, a pharmaceutical composition comprising a therapeutically effective amount of a modified FGF-1 polypeptide is administered in combination with cyclosporine A for treatment of dry eye associated with MGD.

In some embodiments, a pharmaceutical composition comprising a therapeutically effective amount of a modified FGF-1 polypeptide is administered with one or more Rho kinase inhibitors. In some embodiments, a pharmaceutical composition comprising a therapeutically effective amount of a modified FGF-1 polypeptide is administered with one or more additional growth factors, including, but not limited to epidermal growth factor (EGF) and nerve growth factor (NGF). See, e.g., see Joyce et al. (2009) Invest Ophthalmol. Vis Sci. 50:2116-2122, vascular endothelial growth factor (VEGF), transforming growth factor alpha and beta (TGF-alpha and TFG-beta), platelet-derived endothelial growth factor (PD-ECGF), platelet-derived growth factor (PDGF), tumor necrosis factor alpha (TNF-alpha), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxidcsynthase (NOS).

### Kits/Articles of Manufacture

For use in the therapeutic applications described herein, kits and articles of manufacture are also provided herein. Such kits include, in certain embodiments, a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of ophthalmic formulations of the modified FGF-1 polypeptides and pharmaceutical compositions provided herein are contemplated as are a variety of treatments for any disease, disorder, or condition associated with dry eye that would benefit by administration of a modified FGF ore pharmaceutical composition described herein.

For example, the container(s) can include a modified FGF such as a modified FGF-1 having a sequence of SEQ ID NO: 2. The container(s) optionally have a sterile access port. Such kits optionally comprising compounds with an identifying descriptions or labels or instructions relating to their use in the methods described herein.

In some embodiments, a kit may be suitable for or designed to be suitable for an injectable liquid formulation for intraocular delivery. The kit may be designed as a low-volume vial and may comprise a conical insert. In some embodiments, the kit is the dropper bottle. In some embodiments, the dropper bottle may be enabled to provide at least on dose of modified FGF-1 in the injectable formulation. In some embodiments, the dropper bottle further comprises a sterile filter. In some embodiments, the container comprises the syringe. In some embodiments, the syringe comprises a material selected from the group consisting of tuberculin polypropylene and glass. In some embodiments, the syringe is prefilled with an injectable formulation. In some embodiments, the kit may further comprise an electronic control unit. In some embodiments, the electronic control unit enables control of administration of a volume of an injectable formulation according to that described in the preceding sections, wherein the volume is from at least about 10 microliters to about 100 microliters. In some embodiments, the dropper bottle of the kit is enabled to provide at least on dose of modified FGF-1 in the injectable formulation of any one of embodiments described above, or the pharmaceutical composition described anywhere in the disclosure. In some embodiments, the dropper bottle may further comprise a sterile filter. In some embodiments, the container comprises the syringe. In some embodiments, the syringe comprises a material selected from the group consisting of tuberculin polypropylene and glass. In some embodiments, the syringe is prefilled with an injectable formulation according to any one of embodiments described above, or the pharmaceutical composition described anywhere in the disclosure. The kit may further comprise an electronic control unit. In some embodiments, the electronic control unit enables control of administration of a volume of an injectable formulation or a pharmaceutical composition, wherein the volume is from at least about 10 μL to about 100 μL.

In some embodiments, a kit includes one or more additional containers, each with one or more of various materials (such as reagents, optionally in concentrated form, and/or devices) desirable from a commercial and user standpoint for use of a modified FGF described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a modified FGF (FGF-1) polypeptide containing pharmaceutical composition is presented in a pack or dispenser device which contains one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a modified FGF provided herein formulated in a compatible pharmaceutical carrier can also be prepared, placed in an appropriate container, and labeled for treatment of an indicated condition.

### EXAMPLES

These examples are provided for illustrative purposes only and not to limit the scope of the claims provided herein. The starting materials and reagents used in the examples described herein may be synthesized or can be obtained from commercial sources.

### Example 1: Therapeutic Effect of Modified FGF-1 Polypeptides on Meibomian Gland Dysfunction (MGD) Induced-Dry Eye

The study is directed towards the effect of modified FGF-1 polypeptides on treatment of dry eye induced by meibomian gland dysfunction.

The effect of administering a composition comprising the modified FGF-1 polypeptide of sequence of SEQ ID NO: 2 (N-Met-TTHX1114) for treating dry eye disease caused by dysfunction of meibomian gland will be tested in this example. The modified FGF-1 polypeptide comprising the mutations Cys16Ser, Ala66Cys, and Cys117Val will be administered three times daily (t.i.d.) to one eye of mice models with development of MGD (n=3 per dose group) for 7 days. The exemplary formulation used will be 1 mM Histidine, pH=5.8, 5% sorbitol, 0.1% polysorbate 80. To evaluate the effect of the amount of dose, three different doses, including 0.3 μg/eye/dose, 0.9 μg/eye/dose, and 3 μg/eye/dose, will be separately administered to one eye of the mice. At the end of the 7^{th} day, the rate of tear evaporation will be measured using a high-sensitivity microbalance sensor. Statistical test will be two sample t-test assuming equal variances without correction for multiple observations. The dose of 3 μg/eye/dose will decrease the rate of tear evaporation compared to non-treated eye.

### Example 2: Evaluation of Treatment with Modified FGF-1 Polypeptides on Desiccating Stress Model of Dry Eye

A mouse model will be developed by the following procedure: The Lacrimal Functional Unit (LFU) is inhibited with systemic anticholinergic agent in mice that are exposed to a dry, drafty environment. The mouse will be given three daily subcutaneous injections of scopolamine, a short acting anti-cholinergic in order to inhibit tear secretion by the lacrimal glands and conjunctival goblet cells. The mouse will be then maintained in a low humidity environment (20%) and exposed to an air movement for 5-10 days.

The mice will be administered with a composition comprising the modified FGF-1 polypeptide of sequence of SEQ ID NO: 2 (N-Met-TTHX1114). The modified FGF-1 polypeptide comprising the mutations Cys16Ser, Ala66Cys, and Cys117Val will be administered three times daily (t.i.d.) for 7 days. The exemplary formulation used will be 1 mM Histidine, pH=5.8, 5% sorbitol, 0.1% polysorbate 80. To evaluate the effect of the amount of dose, three different doses, including 0.3 μg/eye/dose, 0.9 μg/eye/dose, and 3 μg/eye/dose, will be separately administered to one eye of the mice. At the end of the 7th day, ocular surface damage will be checked and compared with non-treated model. The dose of 3 μg/eye/dose will improve the eye dryness and condition of ocular surface.

TABLE 1
SEQUENCES

| SEQUENCE | No. |
|---|---|
| FNLPPGNYKKPKLLYCSNGGHFLRILPDGTVDGTRDRSDQHIQLQLSAESVGEVY | 1 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MFNLPPGNYKKPKLLYSSNGGHFLRILPDGTVDGTRDRSDQHIQLQLSAESVGEVYIKSTETGQYLCMD | 2 |
| TDGLLYGSQTPNEECLFLERLEENHYNTYISKKHAEKNWFVGLKKNGSVKRGPRTHYGQKAILFLPLPV | |
| SSD | |
| MAEGEITTFTALTEK | 3 |
| ALTEK | 4 |
| LTEK | 5 |
| TEK | 6 |
| EK | 7 |
| K | 8 |
| MALTEKFNLPPGNYKKPKLLYCSNGGHFLRILPDGTVDGTRDRSDQHIQLQLSAESVGEVY | 9 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 10 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 11 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 12 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 13 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 14 |
| YLCMDTDGLLYGSQTPNEECLFLERLEENHYNTYISKKHAEKNWFVGLKKNGSVKRGPRTHYGQKAILF | |
| LPLPVSSD | |
| MLTEKFNLPPGNYKKPKLLYSSNGGHFLRILPDGTVDGTRDRSDQHIQLQLSAESVGEVYIKSTETGQY | 15 |
| LCMDTDGLLYGSQTPNEECLFLERLEENHYNTYISKKHAEKNWFVGLKKNGSVKRGPRTHYGQKAILFL | |
| PLPVSSD | |
| MTEKFNLPPGNYKKPKLLYSSNGGHFLRILPDGTVDGTRDRSDQHIQLQLSAESVGEVYIKSTETGQYL | 16 |
| CMDTDGLLYGSQTPNEECLFLERLEENHYNTYISKKHAEKNWFVGLKKNGSVKRGPRTHYGQKAILFLP | |
| LPVSSD | |
| MEKFNLPPGNYKKPKLLYSSNGGHFLRILPDGTVDGTRDRSDQHIQLQLSAESVGEVYIKSTETGQYLC | 17 |
| MDTDGLLYGSQTPNEECLFLERLEENHYNTYISKKHAEKNWFVGLKKNGSVKRGPRTHYGQKAILFLPL | |
| PVSSD | |
| MKFNLPPGNYKKPKLLYSSNGGHFLRILPDGTVDGTRDRSDQHIQLQLSAESVGEVYIKSTETGQYLCM | 18 |
| DTDGLLYGSQTPNEECLFLERLEENHYNTYISKKHAEKNWFVGLKKNGSVKRGPRTHYGQKAILFLPLP | |
| VSSD | |
| ALTEKFNLPPGNYKKPKLLYCSNGGHFLRILPDGTVDGTRDRSDQHIQLQLSAESVGEVY | 19 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 20 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 21 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 22 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 23 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKFNLPPGNYKKPKLLYSSNGGHFLRILPDGTVDGTRDRSDQHIQLQLSAESVGEVYIKSTETGQY | 24 |
| LCMDTDGLLYGSQTPNEECLFLERLEENHYNTYISKKHAEKNWFVGLKKNGSVKRGPRTHYGQKAILFL | |
| PLPVSSD | |
| LTEKFNLPPGNYKKPKLLYSSNGGHFLRILPDGTVDGTRDRSDQHIQLQLSAESVGEVYIKSTETGQYL | 25 |
| CMDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHFLRILPDGTVDGTRDRSDQHIQLQLSAESVGEVYIKSTETGQYLC | 26 |
| MDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHFLRILPDGTVDGTRDRSDQHIQLQLSAESVGEVYIKSTETGQYLCM | 27 |
| DTDGLLYGSQTPNEECLFLERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCMD | 28 |
| TDGLLYGSQTPNEECLFLERLEENHYNTYISKKHAEKNWFVGLKKNGSVKRGPRTHYGQKAILFLPLPV | |
| SSD | |
| NLPPGNYKKPKLLYCSNGGHFLRILPDGTVDGTRDRSDQHIQLQLSAESVGEVY | 29 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LPPGNYKKPKLLYCSNGGHFLRILPDGTVDGTRDRSDQHIQLQLSAESVGEVY | 30 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PPGNYKKPKLLYCSNGGHFLRILPDGTVDGTRDRSDQHIQLQLSAESVGEVY | 31 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 32 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYSSNGGHFLRILPDGTVDGTRDRSDQHIQLQLSAESVGEVYIKSTETGQYLCMDTD | 33 |
| GLLYGSQTPNEECLFLERLEENHYNTYISKKHAEKNWFVGLKKNGSVKRGPRTHYGQKAILFLPLPVSS | |
| D | |
| LPPGNYKKPKLLYSSNGGHFLRILPDGTVDGTRDRSDQHIQLQLSAESVGEVYIKSTETGQYLCMDTDG | 34 |
| LLYGSQTPNEECLFLERLEENHYNTYISKKHAEKNWFVGLKKNGSVKRGPRTHYGQKAILFLPLPVSSD | |
| PPGNYKKPKLLYSSNGGHFLRILPDGTVDGTRDRSDQHIQLQLSAESVGEVYIKSTETGQYLCMDTDGL | 35 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCMDTDGLL | 36 |
| YGSQTPNEECLFLERLEENHYNTYISKKHAEKNWFVGLKKNGSVKRGPRTHYGQKAILFLPLPVSSD | |
| MNLPPGNYKKPKLLYCSNGGHFLRILPDGTVDGTRDRSDQHIQLQLSAESVGEVY | 37 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLPPGNYKKPKLLYCSNGGHFLRILPDGTVDGTRDRSDQHIQLQLSAESVGEVY | 38 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPPGNYKKPKLLYCSNGGHFLRILPDGTVDGTRDRSDQHIQLQLSAESVGEVY | 39 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPGNYKKPKLLYCSNGGHFLRILPDGTVDGTRDRSDQHIQLQLSAESVGEVY | 40 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MNLPPGNYKKPKLLYSSNGGHFLRILPDGTVDGTRDRSDQHIQLQLSAESVGEVYIKSTETGQYLCMDT | 41 |
| DGLLYGSQTPNEECLFLERLEENHYNTYISKKHAEKNWFVGLKKNGSVKRGPRTHYGQKAILFLPLPVS | |
| SD | |
| MLPPGNYKKPKLLYSSNGGHFLRILPDGTVDGTRDRSDQHIQLQLSAESVGEVYIKSTETGQYLCMDTD | 42 |
| GLLYGSQTPNEECLFLERLEENHYNTYISKKHAEKNWFVGLKKNGSVKRGPRTHYGQKAILFLPLPVSS | |
| D | |
| MPPGNYKKPKLLYSSNGGHFLRILPDGTVDGTRDRSDQHIQLQLSAESVGEVYIKSTETGQYLCMDTDG | 43 |
| LLYGSQTPNEECLFLERLEENHYNTYISKKHAEKNWFVGLKKNGSVKRGPRTHYGQKAILFLPLPVSSD | |
| MPGNYKKPKLLYSSNGGHFLRILPDGTVDGTRDRSDQHIQLQLSAESVGEVYIKSTETGQYLCMDTDGL | 44 |
| LYGSQTPNEECLFLERLEENHYNTYISKKHAEKNWFVGLKKNGSVKRGPRTHYGQKAILFLPLPVSSD | |
| MALTEKNLPPGNYKKPKLLYCSNGGHFLRILPDGTVDGTRDRSDQHIQLQLSAESVGEVY | 45 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKLPPGNYKKPKLLYSSNGGHFLRILPDGTVDGTRDRSDQHIQLQLSAESVGEVYIKSTETGQYL | 46 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGGNYKKPKLLYCSNGGHFLRILPDGTVDGTRDRSDQHIQLQLSAESVGEVY | 47 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKPGNYKKPKLLYCSNGGHFLRILPDGTVDGTRDRSDQHIQLQLSAESVGEVY | 48 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 49 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKNLPPGNYKKPKLLYCSNGGHFLRILPDGTVDGTRDRSDQHIQLQLSAESVGEVY | 50 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKLPPGNYKKPKLLYCSNGGHFLRILPDGTVDGTRDRSDQHIQLQLSAESVGEVY | 51 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPPGNYKKPKLLYCSNGGHFLRILPDGTVDGTRDRSDQHIQLQLSAESVGEVY | 52 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPGNYKKPKLLYCSNGGHFLRILPDGTVDGTRDRSDQHIQLQLSAESVGEVY | 53 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 54 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKNLPPGNYKKPKLLYCSNGGHFLRILPDGTVDGTRDRSDQHIQLQLSAESVGEVY | 55 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKLPPGNYKKPKLLYCSNGGHFLRILPDGTVDGTRDRSDQHIQLQLSAESVGEVY | 56 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKPPGNYKKPKLLYCSNGGHFLRILPDGTVDGTRDRSDQHIQLQLSAESVGEVY | 57 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKPGNYKKPKLLYCSNGGHFLRILPDGTVDGTRDRSDQHIQLQLSAESVGEVY | 58 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 59 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKNLPPGNYKKPKLLYCSNGGHFLRILPDGTVDGTRDRSDQHIQLQLSAESVGEVY | 60 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKLPPGNYKKPKLLYCSNGGHFLRILPDGTVDGTRDRSDQHIQLQLSAESVGEVY | 61 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPPGNYKKPKLLYCSNGGHFLRILPDGTVDGTRDRSDQHIQLQLSAESVGEVY | 62 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPGNYKKPKLLYCSNGGHFLRILPDGTVDGTRDRSDQHIQLQLSAESVGEVY | 63 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 64 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKNLPPGNYKKPKLLYCSNGGHFLRILPDGTVDGTRDRSDQHIQLQLSAESVGEVY | 65 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKLPPGNYKKPKLLYCSNGGHFLRILPDGTVDGTRDRSDQHIQLQLSAESVGEVY | 66 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPPGNYKKPKLLYCSNGGHFLRILPDGTVDGTRDRSDQHIQLQLSAESVGEVY | 67 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPGNYKKPKLLYCSNGGHFLRILPDGTVDGTRDRSDQHIQLQLSAESVGEVY | 68 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYCSNGGHFLRILPDGTVDGTRDRSDQHIQLQLSAESVGEVY | 69 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKLPPGNYKKPKLLYCSNGGHFLRILPDGTVDGTRDRSDQHIQLQLSAESVGEVY | 70 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPPGNYKKPKLLYCSNGGHFLRILPDGTVDGTRDRSDQHIQLQLSAESVGEVY | 71 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 72 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 73 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 74 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 75 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKPPGNYKKPKLLYCSNGGHFLRILPDGTVDGTRDRSDQHIQLQLSAESVGEVY | 76 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 77 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 78 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 79 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 80 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 81 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 82 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 83 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 84 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 85 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 86 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 87 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 88 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 89 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHFLRILPDGTVDGTRDRSDQHIQLQLSAESVGEVY | 90 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KPPGNYKKPKLLYCSNGGHFLRILPDGTVDGTRDRSDQHIQLQLSAESVGEVY | 91 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KPGNYKKPKLLYCSNGGHFLRILPDGTVDGTRDRSDQHIQLQLSAESVGEVY | 92 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYSSNGGHFLRILPDGTVDGTRDRSDQHIQLQLSAESVGEVYIKSTETGQYL | 93 |
| CMDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHFLRILPDGTVDGTRDRSDQHIQLQLSAESVGEVYIKSTETGQYLC | 94 |
| MDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCM | 96 |
| DTDGLLYGSQTPNEECLFLERLEENHYNTYISKKHAEKNWFVGLKKNGSVKRGPRTHYGQKAILFLPLP | |
| VSSD | |
| ALTEKPGNYKKPKLLYSSNGGHFLRILPDGTVDGTRDRSDQHIQLQLSAESVGEVYIKSTETGQYLCMD | 97 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDQHIQLQLSAESVGEVYIKSTETGQYL | 98 |
| CMDTDGLLYGSQTPNEECLFLERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 99 |
| MDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCM | 100 |
| DTDGLLYGSQTPNEECLFLERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCMD | 101 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCMDT | 102 |
| DGLLYGSQTPNEECLFLERLEENHYNTYISKKHAEKNWFVGLKKNGSVKRGPRTHYGQKAILFLPLPVS | |
| SD | |
| TEKFNLPPGNYKKPKLLYSSNGGHFLRILPDGTVDGTRDRSDQHIQLQLSAESVGEVYIKSTETGQYLC | 103 |
| MDTDGLLYGSQTPNEECLFLERLEENHYNTYISKKHAEKNWFVGLKKNGSVKRGPRTHYGQKAILFLPL | |
| PVSSD | |
| TEKNLPPGNYKKPKLLYSSNGGHFLRILPDGTVDGTRDRSDQHIQLQLSAESVGEVYIKSTETGQYLCM | 104 |
| DTDGLLYGSQTPNEECLFLERLEENHYNTYISKKHAEKNWFVGLKKNGSVKRGPRTHYGQKAILFLPLP | |
| VSSD | |
| TEKLPPGNYKKPKLLYSSNGGHFLRILPDGTVDGTRDRSDQHIQLQLSAESVGEVYIKSTETGQYLCMD | 105 |
| TDGLLYGSQTPNEECLFLERLEENHYNTYISKKHAEKNWFVGLKKNGSVKRGPRTHYGQKAILFLPLPV | |
| SSD | |
| TEKPPGNYKKPKLLYSSNGGHFLRILPDGTVDGTRDRSDQHIQLQLSAESVGEVYIKSTETGQYLCMDT | 106 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCMDTD | 107 |
| GLLYGSQTPNEECLFLERLEENHYNTYISKKHAEKNWFVGLKKNGSVKRGPRTHYGQKAILFLPLPVSS | |
| D | |
| EKFNLPPGNYKKPKLLYSSNGGHFLRILPDGTVDGTRDRSDQHIQLQLSAESVGEVYIKSTETGQYLCM | 108 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCMD | 109 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCMDT | 110 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHFLRILPDGTVDGTRDRSDQHIQLQLSAESVGEVYIKSTETGQYLCMDTD | 111 |
| GLLYGSQTPNEECLFLERLEENHYNTYISKKHAEKNWFVGLKKNGSVKRGPRTHYGQKAILFLPLPVSS | |
| D | |
| EKPGNYKKPKLLYSSNGGHFLRILPDGTVDGTRDRSDQHIQLQLSAESVGEVYIKSTETGQYLCMDTDG | 112 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCMD | 113 |
| TDGLLYGSQTPNEECLFLERLEENHYNTYISKKHAEKNWFVGLKKNGSVKRGPRTHYGQKAILFLPLPV | |
| SSD | |
| KNLPPGNYKKPKLLYSSNGGHFLRILPDGTVDGTRDRSDQHIQLQLSAESVGEVYIKSTETGQYLCMDT | 114 |
| DGLLYGSQTPNEECLFLERLEENHYNTYISKKHAEKNWFVGLKKNGSVKRGPRTHYGQKAILFLPLPVS | |
| SD | |
| KLPPGNYKKPKLLYSSNGGHELRILPDGTVDGTRDRSDQHIQLQLSAESVGEVYIKSTETGQYLCMDTD | 115 |
| GLLYGSQTPNEECLFLERLEENHYNTYISKKHAEKNWFVGLKKNGSVKRGPRTHYGQKAILFLPLPVSS | |
| D | |
| KPPGNYKKPKLLYSSNGGHFLRILPDGTVDGTRDRSDQHIQLQLSAESVGEVYIKSTETGQYLCMDTDG | 116 |
| LLYGSQTPNEECLFLERLEENHYNTYISKKHAEKNWFVGLKKNGSVKRGPRTHYGQKAILFLPLPVSSD | |
| KPGNYKKPKLLYSSNGGHFLRILPDGTVDGTRDRSDQHIQLQLSAESVGEVYIKSTETGQYLCMDTDGL | 117 |
| LYGSQTPNEECLFLERLEENHYNTYISKKHAEKNWFVGLKKNGSVKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 118 |
| YLCMDTDGLLYGSQTPNEECLFLERLEENHYNTYISKKHAEKNWFVGLKKNGSVKRGPRTHYGQKAILF | |
| LPLPVSSD | |
| MALTEKNLPPGNYKKPKLLYSSNGGHFLRILPDGTVDGTRDRSDQHIQLQLSAESVGEVYIKSTETGQY | 207 |
| LCMDTDGLLYGSQTPNEECLFLERLEENHYNTYISKKHAEKNWFVGLKKNGSVKRGPRTHYGQKAILFL | |
| PLPVSSD | |
| MALTEKLPPGNYKKPKLLYSSNGGHFLRILPDGTVDGTRDRSDQHIQLQLSAESVGEVYIKSTETGQYL | 119 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGNYKKPKLLYSSNGGHFLRILPDGTVDGTRDRSDQHIQLQLSAESVGEVYIKSTETGQYLC | 120 |
| MDTDGLLYGSQTPNEECLFLERLEENHYNTYISKKHAEKNWFVGLKKNGSVKRGPRTHYGQKAILFLPL | |
| PVSSD | |
| MALTEKPGNYKKPKLLYSSNGGHELRILPDGTVDGTRDRSDQHIQLQLSAESVGEVYIKSTETGQYLCM | 121 |
| DTDGLLYGSQTPNEECLFLERLEENHYNTYISKKHAEKNWFVGLKKNGSVKRGPRTHYGQKAILFLPLP | |
| VSSD | |
| MLTEKFNLPPGNYKKPKLLYSSNGGHFLRILPDGTVDGTRDRSDQHIQLQLSAESVGEVYIKSTETGQY | 122 |
| LCMDTDGLLYGSQTPNEECLFLERLEENHYNTYISKKHAEKNWFVGLKKNGSVKRGPRTHYGQKAILFL | |
| PLPVSSD | |
| MLTEKNLPPGNYKKPKLLYSSNGGHFLRILPDGTVDGTRDRSDQHIQLQLSAESVGEVYIKSTETGQYL | 123 |
| CMDTDGLLYGSQTPNEECLFLERLEENHYNTYISKKHAEKNWFVGLKKNGSVKRGPRTHYGQKAILFLP | |
| LPVSSD | |
| MLTEKLPPGNYKKPKLLYSSNGGHFLRILPDGTVDGTRDRSDQHIQLQLSAESVGEVYIKSTETGQYLC | 124 |
| MDTDGLLYGSQTPNEECLFLERLEENHYNTYISKKHAEKNWFVGLKKNGSVKRGPRTHYGQKAILFLPL | |
| PVSSD | |
| MLTEKPPGNYKKPKLLYSSNGGHFLRILPDGTVDGTRDRSDQHIQLQLSAESVGEVYIKSTETGQYLCM | 125 |
| DTDGLLYGSQTPNEECLFLERLEENHYNTYISKKHAEKNWFVGLKKNGSVKRGPRTHYGQKAILFLPLP | |
| VSSD | |
| MLTEKPGNYKKPKLLYSSNGGHFLRILPDGTVDGTRDRSDQHIQLQLSAESVGEVYIKSTETGQYLCMD | 126 |
| TDGLLYGSQTPNEECLFLERLEENHYNTYISKKHAEKNWFVGLKKNGSVKRGPRTHYGQKAILFLPLPV | |
| SSD | |
| MTEKFNLPPGNYKKPKLLYSSNGGHFLRILPDGTVDGTRDRSDQHIQLQLSAESVGEVYIKSTETGQYL | 127 |
| CMDTDGLLYGSQTPNEECLFLERLEENHYNTYISKKHAEKNWFVGLKKNGSVKRGPRTHYGQKAILFLP | |
| LPVSSD | |
| MTEKNLPPGNYKKPKLLYSSNGGHFLRILPDGTVDGTRDRSDQHIQLQLSAESVGEVYIKSTETGQYLC | 128 |
| MDTDGLLYGSQTPNEECLFLERLEENHYNTYISKKHAEKNWFVGLKKNGSVKRGPRTHYGQKAILFLPL | |
| PVSSD | |
| MTEKLPPGNYKKPKLLYSSNGGHFLRILPDGTVDGTRDRSDQHIQLQLSAESVGEVYIKSTETGQYLCM | 129 |
| DTDGLLYGSQTPNEECLFLERLEENHYNTYISKKHAEKNWFVGLKKNGSVKRGPRTHYGQKAILFLPLP | |
| VSSD | |
| MTEKPPGNYKKPKLLYSSNGGHFLRILPDGTVDGTRDRSDQHIQLQLSAESVGEVYIKSTETGQYLCMD | 130 |
| TDGLLYGSQTPNEECLFLERLEENHYNTYISKKHAEKNWFVGLKKNGSVKRGPRTHYGQKAILFLPLPV | |
| SSD | |
| MTEKPGNYKKPKLLYSSNGGHFLRILPDGTVDGTRDRSDQHIQLQLSAESVGEVYIKSTETGQYLCMDT | 131 |
| DGLLYGSQTPNEECLFLERLEENHYNTYISKKHAEKNWFVGLKKNGSVKRGPRTHYGQKAILFLPLPVS | |
| SD | |
| MEKFNLPPGNYKKPKLLYSSNGGHFLRILPDGTVDGTRDRSDQHIQLQLSAESVGEVYIKSTETGQYLC | 132 |
| MDTDGLLYGSQTPNEECLFLERLEENHYNTYISKKHAEKNWFVGLKKNGSVKRGPRTHYGQKAILFLPL | |
| PVSSD | |
| MEKNLPPGNYKKPKLLYSSNGGHFLRILPDGTVDGTRDRSDQHIQLQLSAESVGEVYIKSTETGQYLCM | 133 |
| DTDGLLYGSQTPNEECLFLERLEENHYNTYISKKHAEKNWFVGLKKNGSVKRGPRTHYGQKAILFLPLP | |
| VSSD | |
| MEKLPPGNYKKPKLLYSSNGGHFLRILPDGTVDGTRDRSDQHIQLQLSAESVGEVYIKSTETGQYLCMD | 134 |
| TDGLLYGSQTPNEECLFLERLEENHYNTYISKKHAEKNWFVGLKKNGSVKRGPRTHYGQKAILFLPLPV | |
| SSD | |
| MEKPPGNYKKPKLLYSSNGGHFLRILPDGTVDGTRDRSDQHIQLQLSAESVGEVYIKSTETGQYLCMDT | 135 |
| DGLLYGSQTPNEECLFLERLEENHYNTYISKKHAEKNWFVGLKKNGSVKRGPRTHYGQKAILFLPLPVS | |
| SD | |
| MEKPGNYKKPKLLYSSNGGHFLRILPDGTVDGTRDRSDQHIQLQLSAESVGEVYIKSTETGQYLCMDTD | 136 |
| GLLYGSQTPNEECLFLERLEENHYNTYISKKHAEKNWFVGLKKNGSVKRGPRTHYGQKAILFLPLPVSS | |
| D | |
| MKFNLPPGNYKKPKLLYSSNGGHFLRILPDGTVDGTRDRSDQHIQLQLSAESVGEVYIKSTETGQYLCM | 137 |
| DTDGLLYGSQTPNEECLFLERLEENHYNTYISKKHAEKNWFVGLKKNGSVKRGPRTHYGQKAILFLPLP | |
| VSSD | |
| MKNLPPGNYKKPKLLYSSNGGHFLRILPDGTVDGTRDRSDQHIQLQLSAESVGEVYIKSTETGQYLCMD | 138 |
| TDGLLYGSQTPNEECLFLERLEENHYNTYISKKHAEKNWFVGLKKNGSVKRGPRTHYGQKAILFLPLPV | |
| SSD | |
| MKLPPGNYKKPKLLYSSNGGHFLRILPDGTVDGTRDRSDQHIQLQLSAESVGEVYIKSTETGQYLCMDT | 139 |
| DGLLYGSQTPNEECLFLERLEENHYNTYISKKHAEKNWFVGLKKNGSVKRGPRTHYGQKAILFLPLPVS | |
| SD | |
| MKPPGNYKKPKLLYSSNGGHFLRILPDGTVDGTRDRSDQHIQLQLSAESVGEVYIKSTETGQYLCMDTD | 140 |
| GLLYGSQTPNEECLFLERLEENHYNTYISKKHAEKNWFVGLKKNGSVKRGPRTHYGQKAILFLPLPVSS | |
| D | |
| MKPGNYKKPKLLYSSNGGHFLRILPDGTVDGTRDRSDQHIQLQLSAESVGEVYIKSTETGQYLCMDTDG | 141 |
| LLYGSQTPNEECLFLERLEENHYNTYISKKHAEKNWFVGLKKNGSVKRGPRTHYGQKAILFLPLPVSSD | |
| FNLPPGNYKKPKLLYCSNGGHFLRILPDGTVDGTRDRSDQHIQLQLSAESVGEVY | 142 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYCSNGGHFLRILPDGTVDGTRDRSDQHIQLQLSAESVGEVY | 143 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| PPGNYKKPKLLYCSNGGHFLRILPDGTVDGTRDRSDQHIQLQLSAESVGEVY | 144 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 145 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 146 |
| DGLLYGSQTPNEECLFLERLEENHYNTYISKKHAEKNWFVGLKKNGSVKRGPRTHYGQKAILFLPLPVS | |
| SD | |
| NLPPGNYKKPKLLYSSNGGHFLRILPDGTVDGTRDRSDQHIQLQLSAESVGEVYIKSTETGQYLCXDTD | 147 |
| GLLYGSQTPNEECLFLERLEENHYNTYISKKHAEKNWFVGLKKNGSVKRGPRTHYGQKAILFLPLPVSS | |
| D | |
| PPGNYKKPKLLYSSNGGHFLRILPDGTVDGTRDRSDQHIQLQLSAESVGEVYIKSTETGQYLCXDTDGL | 148 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCXDTDGLL | 149 |
| YGSQTPNEECLFLERLEENHYNTYISKKHAEKNWFVGLKKNGSVKRGPRTHYGQKAILFLPLPVSSD | |
| ALTEKNLPPGNYKKPKLLYCSNGGHFLRILPDGTVDGTRDRSDQHIQLQLSAESVGEVY | 150 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKLPPGNYKKPKLLYSSNGGHFLRILPDGTVDGTRDRSDQHIQLQLSAESVGEVYIKSTETGQYLC | 151 |
| XDTDGLLYGSQTPNEECLFLERLEENHYNTYISKKHAEKNWFVGLKKNGSVKRGPRTHYGQKAILFLPL | |
| PVSSD | |
| ALTEKPPGGNYKKPKLLYCSNGGHFLRILPDGTVDGTRDRSDQHIQLQLSAESVGEVY | 152 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 153 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 154 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 155 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 156 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPPGNYKKPKLLYCSNGGHFLRILPDGTVDGTRDRSDQHIQLQLSAESVGEVY | 157 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 158 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 159 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 160 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 161 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 162 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 163 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 164 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 165 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 166 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 167 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 168 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 169 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 170 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHFLRILPDGTVDGTRDRSDQHIQLQLSAESVGEVY | 171 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KPPGNYKKPKLLYCSNGGHFLRILPDGTVDGTRDRSDQHIQLQLSAESVGEVY | 172 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KPGNYKKPKLLYCSNGGHFLRILPDGTVDGTRDRSDQHIQLQLSAESVGEVY | 173 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 174 |
| DGLLYGSQTPNEECLFLERLEENHYNTYISKKHAEKNWFVGLKKNGSVKRGPRTHYGQKAILFLPLPVS | |
| SD | |
| ALTEKFNLPPGNYKKPKLLYSSNGGHFLRILPDGTVDGTRDRSDQHIQLQLSAESVGEVYIKSTETGQY | 175 |
| LCXDTDGLLYGSQTPNEECLFLERLEENHYNTYISKKHAEKNWFVGLKKNGSVKRGPRTHYGQKAILFL | |
| PLPVSSD | |
| LTEKFNLPPGNYKKPKLLYSSNGGHFLRILPDGTVDGTRDRSDQHIQLQLSAESVGEVYIKSTETGQYL | 176 |
| CXDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHFLRILPDGTVDGTRDRSDQHIQLQLSAESVGEVYIKSTETGQYLC | 177 |
| XDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHELRILPDGTVDGTRDRSDQHIQLQLSAESVGEVYIKSTETGQYLCX | 178 |
| DTDGLLYGSQTPNEECLFLERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCXD | 179 |
| TDGLLYGSQTPNEECLFLERLEENHYNTYISKKHAEKNWFVGLKKNGSVKRGPRTHYGQKAILFLPLPV | |
| SSD | |
| KFNLPPGNYKKPKLLYSSNGGHFLRILPDGTVDGTRDRSDQHIQLQLSAESVGEVYIKSTETGQYLCXD | 180 |
| TDGLLYGSQTPNEECLFLERLEENHYNTYISKKHAEKNWFVGLKKNGSVKRGPRTHYGQKAILFLPLPV | |
| SSD | |
| ALTEKNLPPGNYKKPKLLYSSNGGHFLRILPDGTVDGTRDRSDQHIQLQLSAESVGEVYIKSTETGQYL | 181 |
| CXDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHFLRILPDGTVDGTRDRSDQHIQLQLSAESVGEVYIKSTETGQYLC | 182 |
| XDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCX | 183 |
| DTDGLLYGSQTPNEECLFLERLEENHYNTYISKKHAEKNWFVGLKKNGSVKRGPRTHYGQKAILFLPLP | |
| VSSD | |
| ALTEKPGNYKKPKLLYSSNGGHFLRILPDGTVDGTRDRSDQHIQLQLSAESVGEVYIKSTETGQYLCXD | 184 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDQHIQLQLSAESVGEVYIKSTETGQYL | 185 |
| CXDTDGLLYGSQTPNEECLFLERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 186 |
| XDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCX | 187 |
| DTDGLLYGSQTPNEECLFLERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCXD | 188 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCXDT | 189 |
| DGLLYGSQTPNEECLFLERLEENHYNTYISKKHAEKNWFVGLKKNGSVKRGPRTHYGQKAILFLPLPVS | |
| SD | |
| TEKFNLPPGNYKKPKLLYSSNGGHFLRILPDGTVDGTRDRSDQHIQLQLSAESVGEVYIKSTETGQYLC | 190 |
| XDTDGLLYGSQTPNEECLFLERLEENHYNTYISKKHAEKNWFVGLKKNGSVKRGPRTHYGQKAILFLPL | |
| PVSSD | |
| TEKNLPPGNYKKPKLLYSSNGGHFLRILPDGTVDGTRDRSDQHIQLQLSAESVGEVYIKSTETGQYLCX | 191 |
| DTDGLLYGSQTPNEECLFLERLEENHYNTYISKKHAEKNWFVGLKKNGSVKRGPRTHYGQKAILFLPLP | |
| VSSD | |
| TEKLPPGNYKKPKLLYSSNGGHFLRILPDGTVDGTRDRSDQHIQLQLSAESVGEVYIKSTETGQYLCXD | 192 |
| TDGLLYGSQTPNEECLFLERLEENHYNTYISKKHAEKNWFVGLKKNGSVKRGPRTHYGQKAILFLPLPV | |
| SSD | |
| TEKPPGNYKKPKLLYSSNGGHFLRILPDGTVDGTRDRSDQHIQLQLSAESVGEVYIKSTETGQYLCXDT | 193 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCXDTD | 194 |
| GLLYGSQTPNEECLFLERLEENHYNTYISKKHAEKNWFVGLKKNGSVKRGPRTHYGQKAILFLPLPVSS | |
| D | |
| EKFNLPPGNYKKPKLLYSSNGGHFLRILPDGTVDGTRDRSDQHIQLQLSAESVGEVYIKSTETGQYLCX | 195 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCXD | 196 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCXDT | 197 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHFLRILPDGTVDGTRDRSDQHIQLQLSAESVGEVYIKSTETGQYLCXDTD | 198 |
| GLLYGSQTPNEECLFLERLEENHYNTYISKKHAEKNWFVGLKKNGSVKRGPRTHYGQKAILFLPLPVSS | |
| D | |
| EKPGNYKKPKLLYSSNGGHFLRILPDGTVDGTRDRSDQHIQLQLSAESVGEVYIKSTETGQYLCXDTDG | 199 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCXD | 200 |
| TDGLLYGSQTPNEECLFLERLEENHYNTYISKKHAEKNWFVGLKKNGSVKRGPRTHYGQKAILFLPLPV | |
| SSD | |
| KNLPPGNYKKPKLLYSSNGGHFLRILPDGTVDGTRDRSDQHIQLQLSAESVGEVYIKSTETGQYLCXDT | 201 |
| DGLLYGSQTPNEECLFLERLEENHYNTYISKKHAEKNWFVGLKKNGSVKRGPRTHYGQKAILFLPLPVS | |
| SD | |
| KLPPGNYKKPKLLYSSNGGHFLRILPDGTVDGTRDRSDQHIQLQLSAESVGEVYIKSTETGQYLCXDTD | 202 |
| GLLYGSQTPNEECLFLERLEENHYNTYISKKHAEKNWFVGLKKNGSVKRGPRTHYGQKAILFLPLPVSS | |
| D | |
| KPPGNYKKPKLLYSSNGGHFLRILPDGTVDGTRDRSDQHIQLQLSAESVGEVYIKSTETGQYLCXDTDG | 203 |
| LLYGSQTPNEECLFLERLEENHYNTYISKKHAEKNWFVGLKKNGSVKRGPRTHYGQKAILFLPLPVSSD | |
| KPGNYKKPKLLYSSNGGHFLRILPDGTVDGTRDRSDQHIQLQLSAESVGEVYIKSTETGQYLCXDTDGL | 204 |
| LYGSQTPNEECLFLERLEENHYNTYISKKHAEKNWFVGLKKNGSVKRGPRTHYGQKAILFLPLPVSSD | |
| FNLPPGNYKKPVLLYCSNGGHFLRILPDGTVDGTRDRSDQHIQLQLSAESVGEVYIKSTETGQYLAMDT | 205 |
| DGLLYGSQTPNEECLFLERLEENHYNTYISKKHAEK | |
| NWFVGLKKNGSVKRGPRTHYGQKAILFLVLPVSSD | |
| FNLPPGNYKKPKLLYSSNGGHFLRILPDGTVDGTRDRSDQHIQLQLSAESVGEVYIKSTETGQYLCMDT | 206 |
| DGLLYGSQTPNEECLFLERLEENHYNTYISKKHAEKNWFVGLKKNGSVKRGPRTHYGQKAILFLPLPVS | |
| SD | |

1. A method of treating dry eye in a subject in need thereof, the method comprising: administering to the subject a pharmaceutical composition comprising a therapeutically effective amount of a modified FGF-1 polypeptide, wherein the modified FGF-1 polypeptide comprises:
a) a wild-type FGF-1 sequence of SEQ ID NO: 1 with mutations of Cys16Ser, Ala66Cys, and Cys117Val; and
b) an N-terminal methionine residue positioned upstream to the first residue of SEQ ID NO:1.

2. The method of claim 1, wherein the dry eye is caused by meibomian gland dysfunction, lacrimal gland insufficiency, lacrimal duct obstruction, reflex hyposecretion, Sjogren's syndrome, eyelid aperture disorder, blink disorder, or an ocular surface disorder.

3. The method of claim 2, wherein the dye eye is caused by the Sjogren's syndrome, wherein the Sjogren's syndrome is a primary Sjogren's syndrome or a secondary Sjogren's syndrome.

4. The method of claim 1, wherein the dry eye is caused by an autoimmune disease.

5. The method of claim 4, wherein the autoimmune disease comprises systemic lupus erythematosus, scleroderma, or rheumatoid arthritis.

6. The method of claim 1, wherein the dry eye is caused by a disease selected from the group comprising GVHD-induced dry eye, radiation-induced dry eye, keratoconjunctivitis sicca, Stevens-Johnson syndrome, lacrimo-auriculo-dento-digital syndrome, ocular cicatricial pemphigoid, ocular surface infection, Riley-Day syndrome, congenital alacrima, nutritional disorders or deficiencies, a glandular destruction, a tissue destruction, an immunodeficient disorder, an inability to blink in comatose patient, a systemic disease, a virus infection, a bacterial infection, a skin disease, an environmental exposure to airborne particulates, smoke, or smog, contact lens intolerance, and a long-term exposure to a display device.

7. The method of claim 6, wherein the dry eye is caused by the systemic disease, wherein the systemic disease comprises Parkinson's disease, diabetes, or thyroid disease.

8. The method of claim 6, wherein the dry eye is caused by the virus infection, wherein the virus comprises human T-cell lymphotropic virus (HTLV), human immunodeficiency virus (HIV), Epstein-Barr virus (EBV), hepatitis C virus (HCV), Influenza, or Measles virus.

9. The method of claim 6, wherein the dry eye is caused by the skin disease, wherein the skin disease comprises rosacea, blepharitis, or pruritus.

10. The method of claim 1, wherein the pharmaceutical composition comprises about 5% (w/v) of a humectant.

11. The method of claim 10, wherein the humectant is selected from the group comprising Glycerin, Maltitol, Propylene Glycol, Sorbitol, and Triacetin.

12. The method of claim 1, wherein the pharmaceutical composition comprises about 0.1% (w/v) of a surfactant.

13. The method of claim 12, wherein the surfactant comprises polysorbate 80 or polysorbate 20.

14. The method of claim 1, wherein the pharmaceutical composition comprises about 1 mM or about 10 mM citrate or Histidine.

15. The method of claim 1, wherein the pharmaceutical composition comprises about 1 mM or 10 mM citrate or Histidine, about 5% (w/v) sorbitol, and about 0.1% (w/v) polysorbate 80.

16. (canceled)

17. (canceled)

18. (canceled)

19. The method of claim 1, wherein the modified FGF-1 polypeptide comprises the sequence of SEQ ID NO: 2.

20. (canceled)

21. (canceled)

22. (canceled)

23. (canceled)

24. (canceled)

25. (canceled)

26. (canceled)

27. (canceled)

28. The method of claim 1, wherein the administering reduces or eliminates a clinical symptom associated with dry eye, wherein the clinical symptom comprises ocular dryness, grittiness, irritation, mucoid discharge, hyperemia, photophobia, or blurry vision.

29. (canceled)

30. (canceled)

31. (canceled)

32. (canceled)

33. The method of claim 1, wherein the pharmaceutical composition is administered by topical application.

34. The method of claim 1, wherein the pharmaceutical composition is administered as an eye drop.

35. The method of claim 15, wherein the pharmaceutical composition has a pH of about 5.8.
